(19) United States
(12) Patent Application Publication
(10) Pub. No.: US 2014/0030321 A1
(43) Pub. Date: Jan. 30, 2014

(54) SOLID PHARMACEUTICAL COMPOSITION

(71) Applicants: Allen Ritter, Lafayette, IN (US); Amy C. Williams, Glen Allen, VA (US); Lars Waldmann, Hyde Park, MA (US); Huamin Zhang, West Lafayette, IN (US)

(72) Inventors: Allen Ritter, Lafayette, IN (US); Amy C. Williams, Glen Allen, VA (US); Lars Waldmann, Hyde Park, MA (US); Huamin Zhang, West Lafayette, IN (US)

(73) Assignee: ENDOCYTE, INC., West Lafayette, IN (US)

(21) Appl. No.: 14/111,099

(22) PCT Filed: Apr. 12, 2012
(86) PCT No.: PCT/US12/33312

§ 371 Date: Oct. 10, 2013

Related U.S. Application Data
(60) Provisional application No. 61/474,428, filed on Apr. 12, 2011.

(51) Int. Cl. A61K 38/08; A61K 9/10; A61J 1/00; A61K 31/704

(52) U.S. Cl. CPC A61K 38/08; A61K 31/704; A61K 9/10; A61J 1/00
USPC 424/450; 514/21.8; 604/403

(57) ABSTRACT

The invention described herein pertains to a solid pharmaceutical composition comprising EC145 for reconstitution to provide a solution for intravenous injection, particularly to a lyophilized solid pharmaceutical composition comprising EC145 which has adequate stability for storage at ambient temperature and which is capable of redissolving in an aqueous diluent prior to administration, as well as a process for its manufacture, drug products comprising the composition and methods for using the composition for treating cancer.

### CROSS-REFERENCE TO RELATED APPLICATIONS

This application claims priority under 35 U.S.C. §119(e) to U.S. Provisional Application Ser. No. 61/474,428, filed Apr. 12, 2011, which is expressly incorporated by reference herein.

### TECHNICAL FIELD

The invention described herein pertains to a solid pharmaceutical composition comprising EC145 for reconstitution to provide a solution for intravenous injection, particularly to a lyophilized solid pharmaceutical composition comprising EC145 which has adequate stability for storage at ambient temperature and which is capable of redissolving in an aqueous diluent prior to administration, as well as a process for its manufacture, drug products comprising the composition and methods for using the composition for treating cancer.

### BACKGROUND AND SUMMARY OF THE INVENTION

Folate-targeted drugs have been developed and are being tested in clinical trials as cancer therapeutics. EC145, also known as vintafolide, comprises a highly potent vinca alkaloid cytotoxic compound, desacetylvinblastine hydrazide (DAVLBH), conjugated to folate. The EC145 molecule targets the folate receptor found at high levels on the surface of epithelial tumors, including non-small cell lung carcinomas (NSCLC), ovarian, endometrial and renal cancers, and others, including fallopian tube and primary peritoneal carcinomas. It is believed that EC145 binds to tumors that express the folate receptor delivering the vinca moiety directly to cancer cells while avoiding normal tissue. Thus, upon binding, EC145 enters the cancer cell via endocytosis, releases DAVLBH and causes cell death or inhibits cell function. EC145 has the following formula

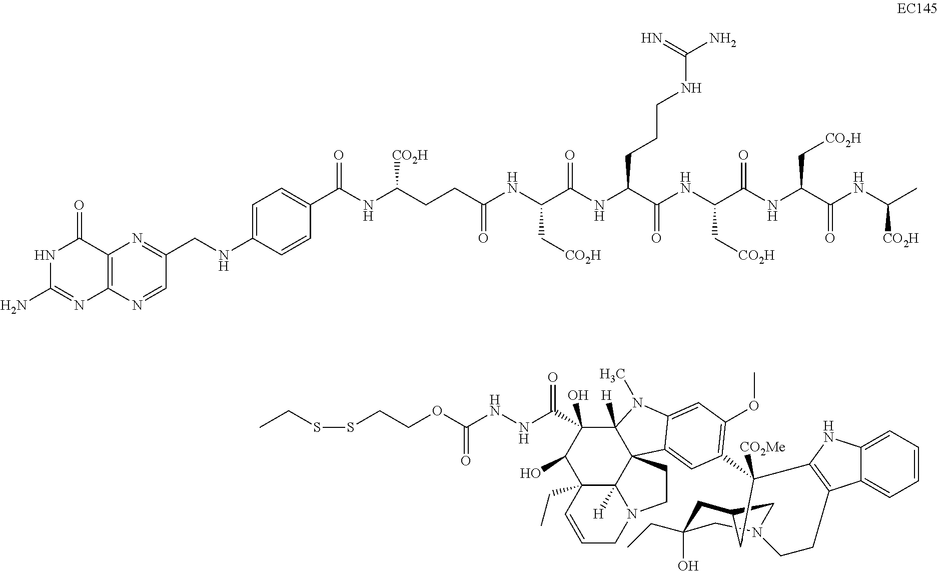

and has been accorded the Chemical Abstracts Registry Number 742092-03-1. As used herein, according to the context, the term EC145 means the compound, or a pharmaceutically acceptable salt thereof; and the compound may be present in a solid, solution or suspension in an ionized form, including a protonated form.

EC145 is disclosed in U.S. Pat. No. 7,601,332; and particular uses and an aqueous liquid pH 7.4, phosphate-buffered formulation for intravenous administration are disclosed in WO 2011/014821. As described in WO 2011/014821, it is necessary to store the aqueous liquid formulation in the frozen state to ensure its stability. To avoid this necessity, a formulation is needed which has adequate stability at ambient temperature.

As one aspect of the invention described herein, there is provided a pharmaceutical composition of EC145 which is a lyophilized solid which has adequate stability for storage at ambient temperature and which is capable of redissolving in an aqueous diluent prior to administration.

In another aspect of the invention, there is provided a pharmaceutical composition of EC145 which is an X-ray amorphous solid which has adequate stability for storage at ambient temperature and which is capable of redissolving in an aqueous diluent prior to administration.

### BRIEF DESCRIPTION OF THE DRAWINGS

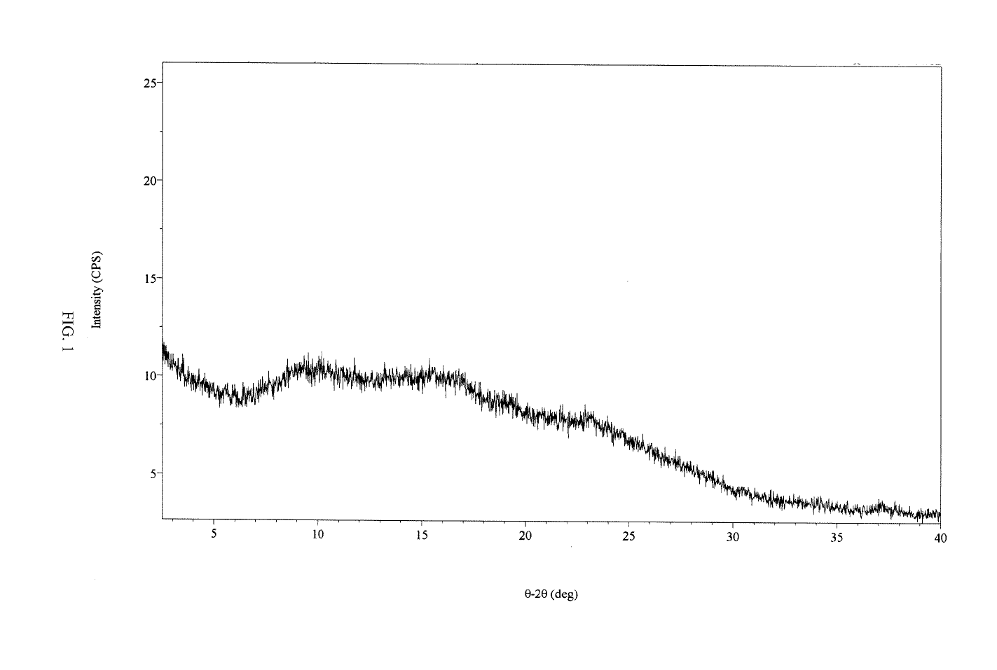

In FIG. 1 is shown the X-ray powder diffraction (XRPD) pattern of lyophilized EC145.

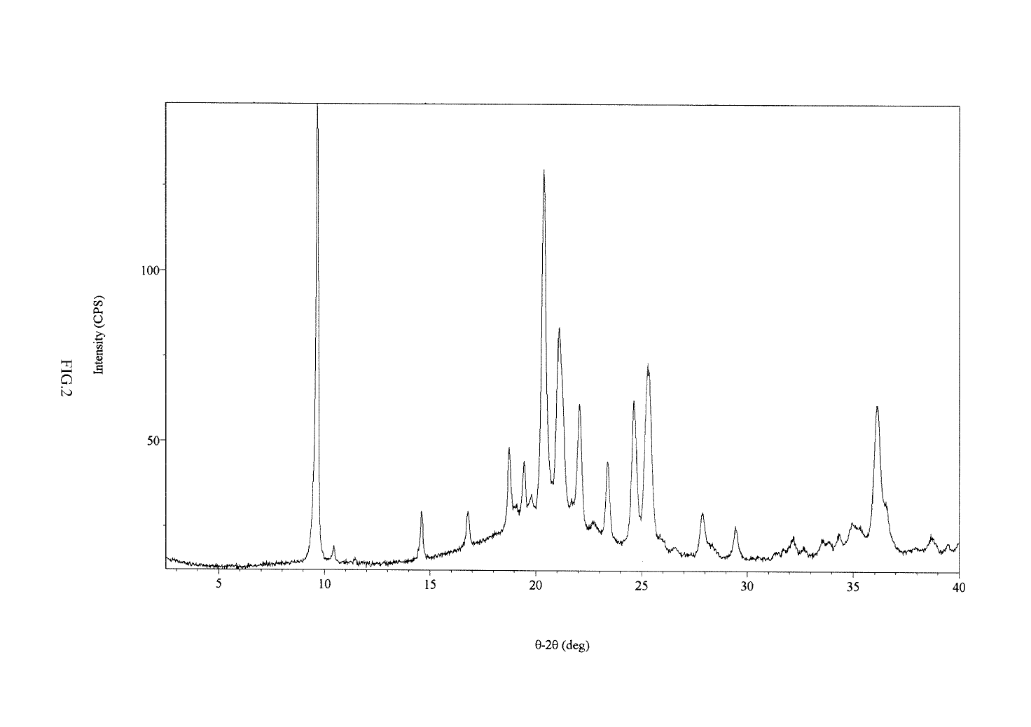

In FIG. 2 is shown the X-ray powder diffraction pattern of a lyophilized, pH 6.2 citrate-buffered EC145 formulation with 3% mannitol.

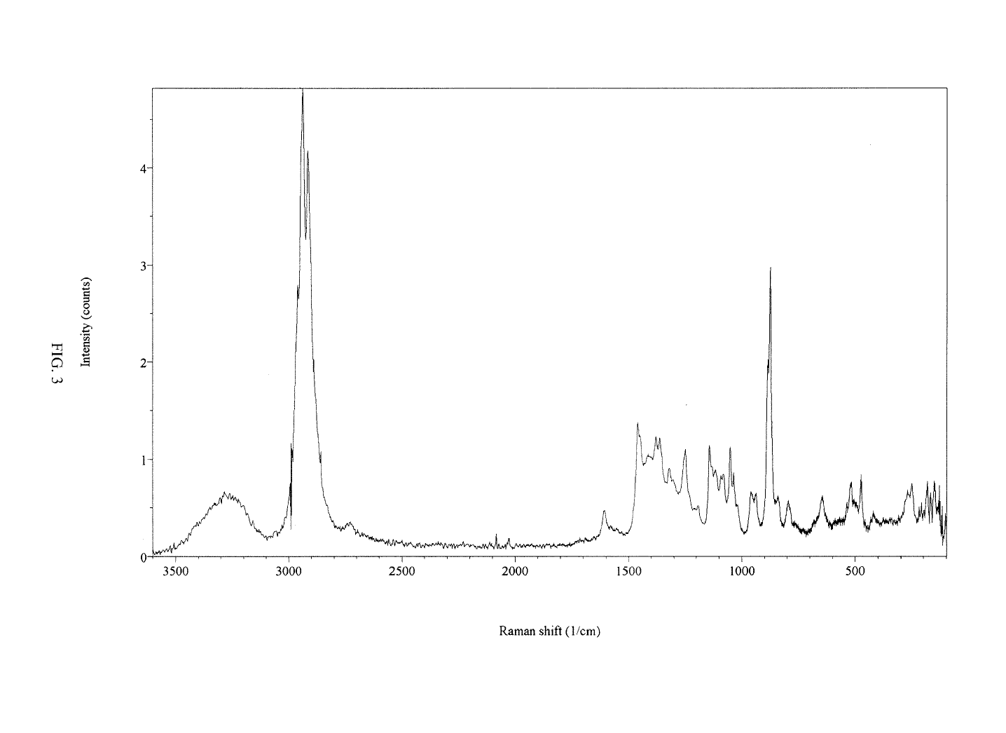

In FIG. 3 is shown the Raman spectrum of a lyophilized, pH 6.2 citrate-buffered EC145 formulation with 3% mannitol.

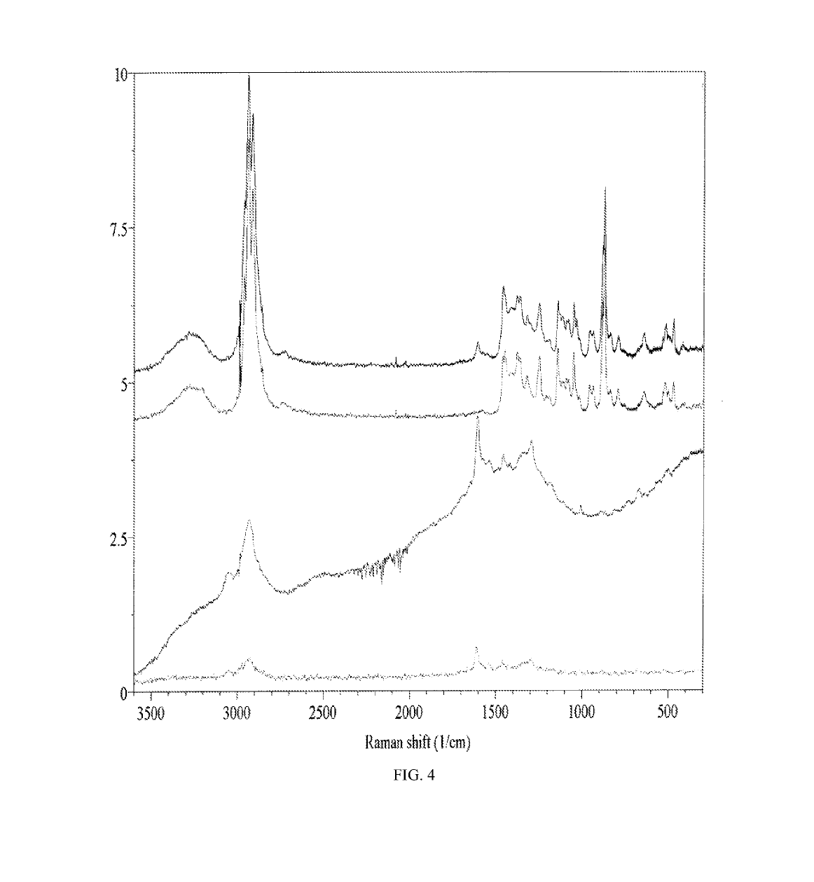

In FIG. 4 is shown the overlay of the Raman spectra of a lyophilized, pH 6.2 citrate-buffered EC145 formulation with 3% mannitol (top), a lyophilized placebo formulation (second from top), lyophilized EC145 (second from bottom), and a prior batch of lyophilized EC145 (bottom).

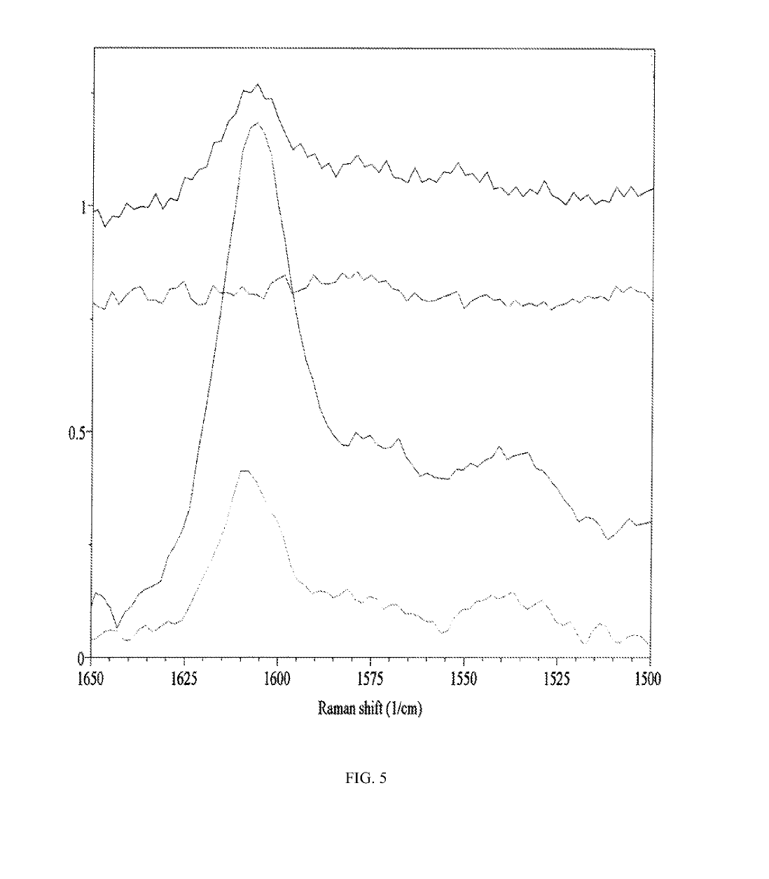

In FIG. 5 is shown the overlay of the expansion from 1500 to 1650 cm⁻¹ of the Raman spectra of a lyophilized, pH 6.2 citrate-buffered EC145 formulation with 3% mannitol (top), a lyophilized placebo formulation (second from top), lyophilized EC145 (second from bottom), and a prior batch of lyophilized EC145 (bottom).

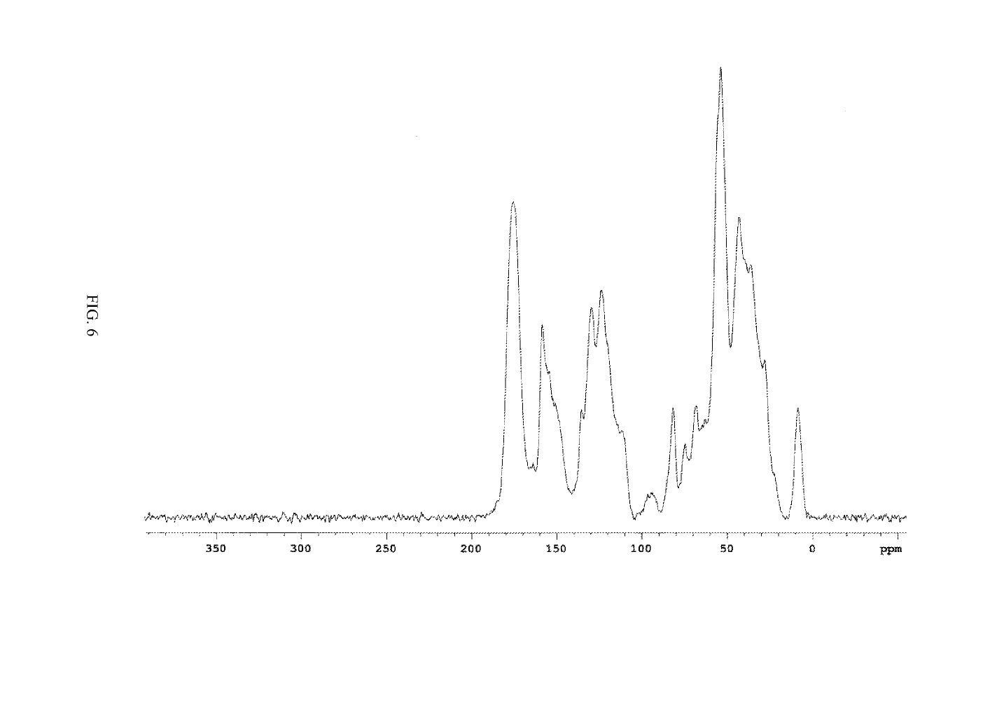

In FIG. 6 is shown the ¹³C CP/MAS NMR spectrum of lyophilized EC145, externally referenced to glycine at 176.5 ppm.

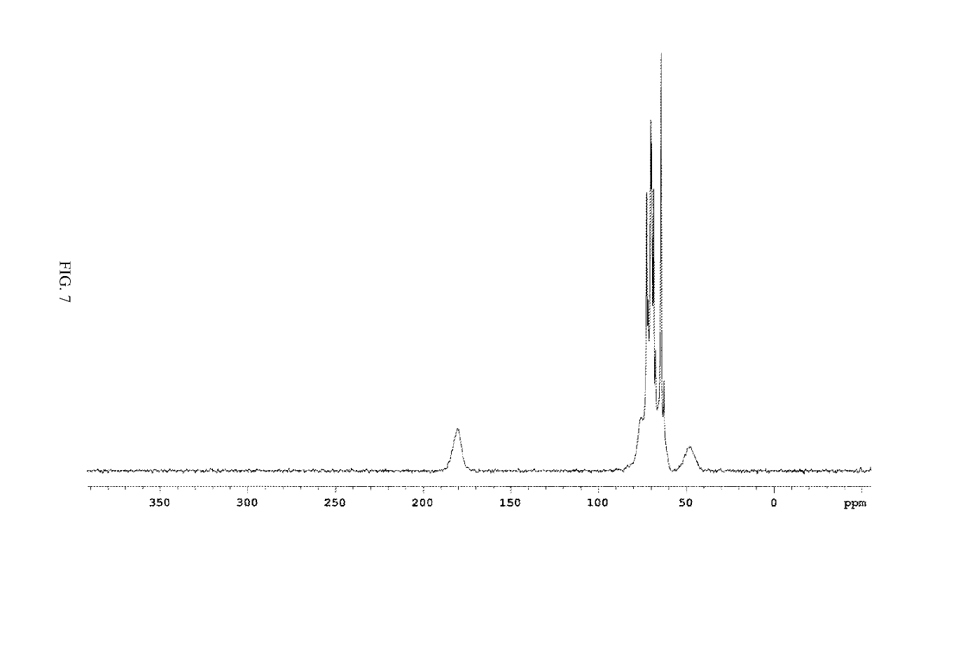

In FIG. 7 is shown the ¹³C CP/MAS NMR spectrum of a lyophilized, pH 6.2 citrate-buffered EC145 formulation with 3% mannitol.

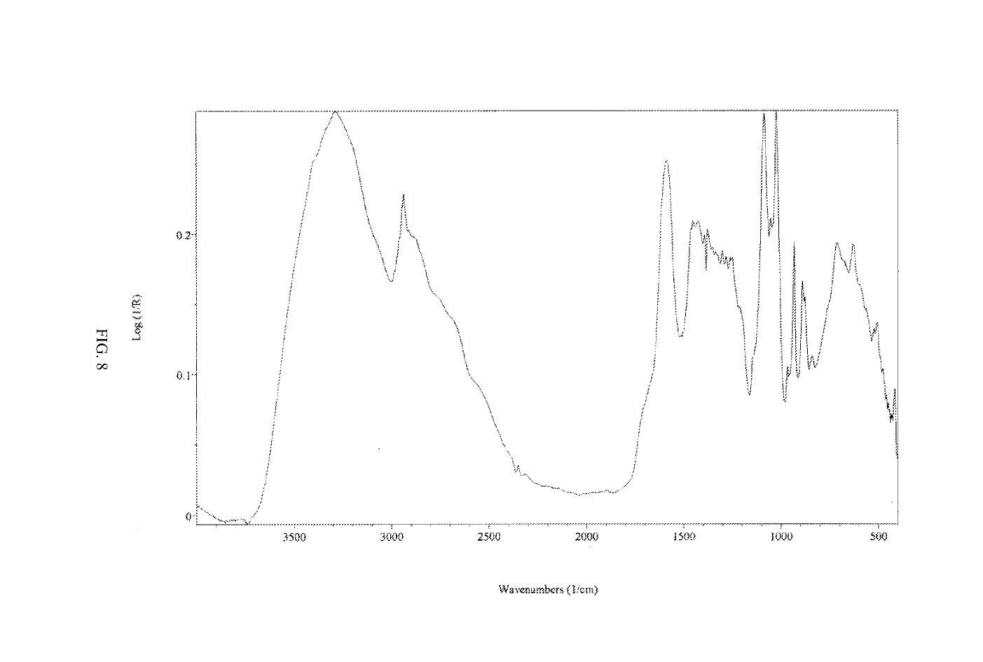

In FIG. 8 is shown the infra red spectrum (DRIFTS) of a lyophilized, pH 6.2 citrate-buffered EC145 formulation with 3% mannitol.

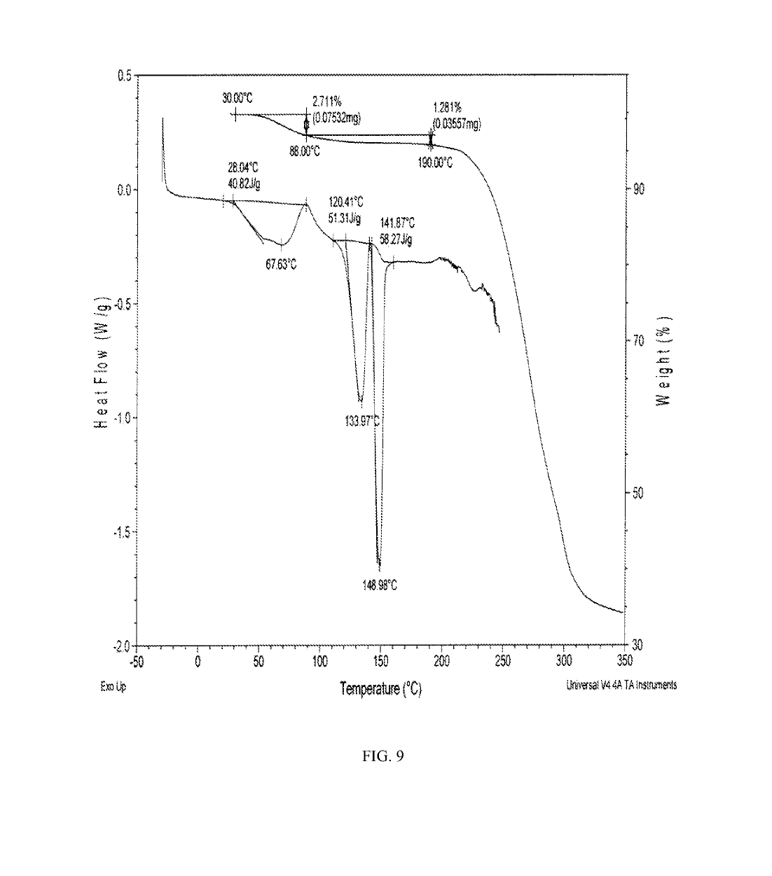

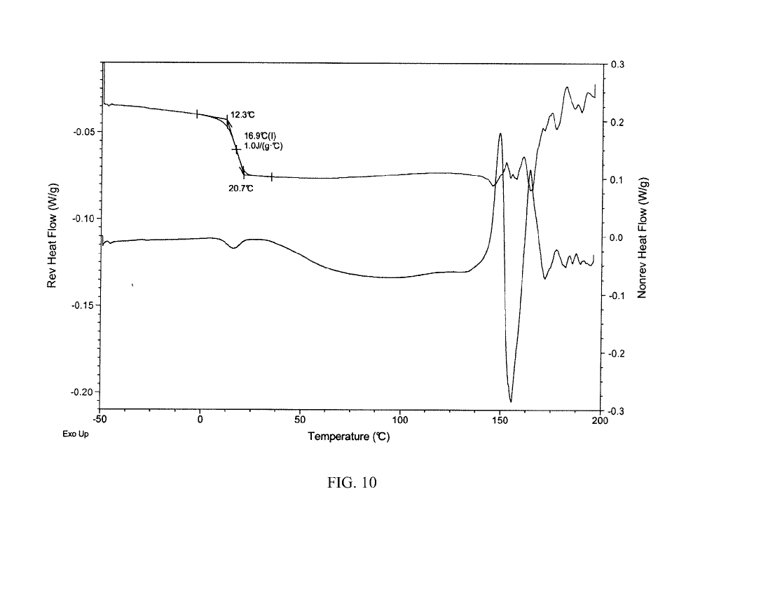

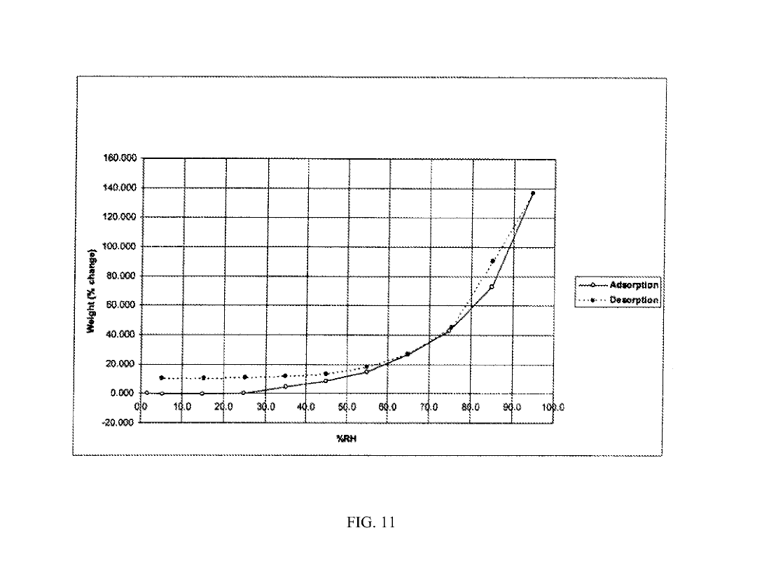

In FIG. 9 are shown the DSC (left scale, left and lower curve) and TGA (right scale, upper and right curve) thermograms of a lyophilized, pH 6.2 citrate-buffered EC145 formulation with 3% mannitol. In FIG. 10 are shown the curves of a modulated DSC thermogram of an EC145 solid dispersion prepared in 50 mM citrate buffer/pH 6.2 containing 1.4 mg/mLEC145 and 3% glucose. In FIG. 11 is shown the dynamic vapor sorption/desorption isotherm of an EC145 solid dispersion prepared in 50 mM citrate buffer/pH 6.2 containing 1.4 mg/mLEC145 and 3% glucose.

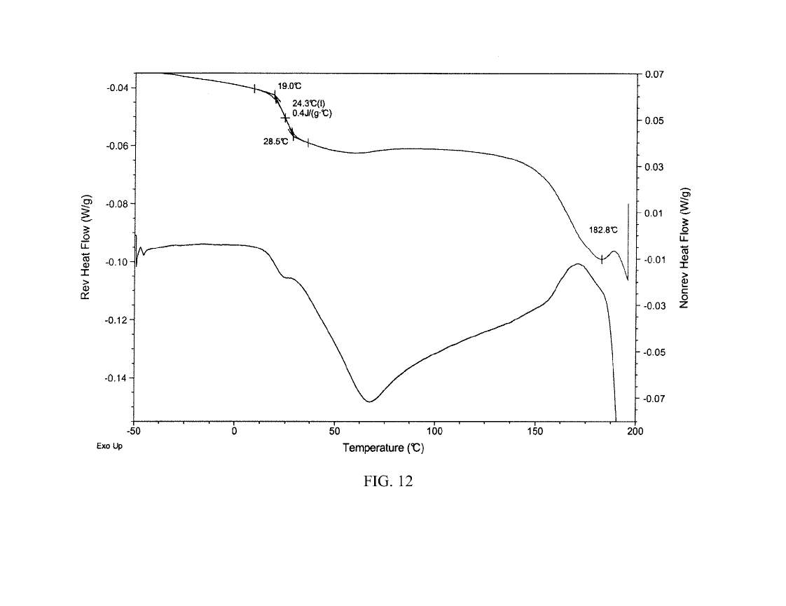

In FIG. 12 are shown the curves of a modulated DSC thermogram of an EC145 solid dispersion prepared in 50 mM citrate buffer/pH 6.2 containing 1.4 mg/mL EC145 and 3% glycine.

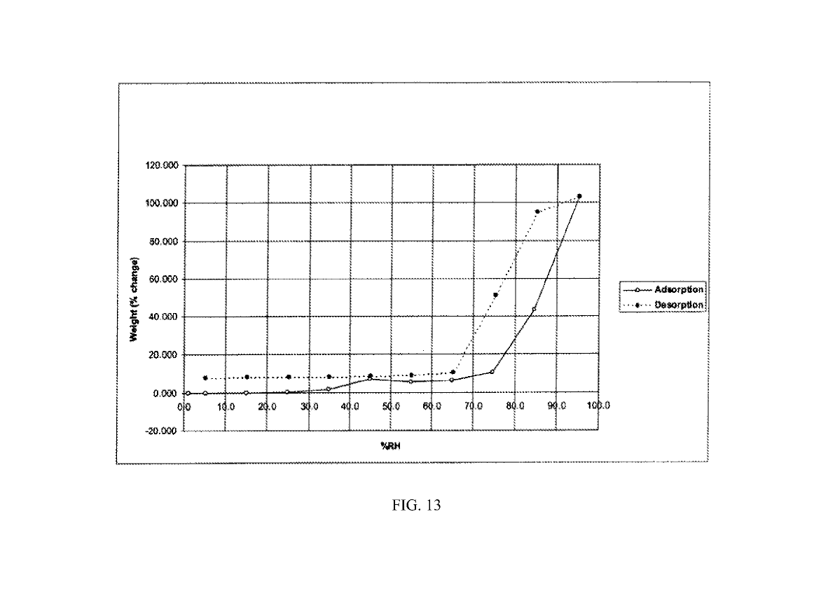

In FIG. 13 is shown the dynamic vapor sorption/desorption isotherm of an EC145 solid dispersion prepared in 50 mM citrate buffer/pH 6.2 containing 1.4 mg/mL EC145 and 3% glycine.

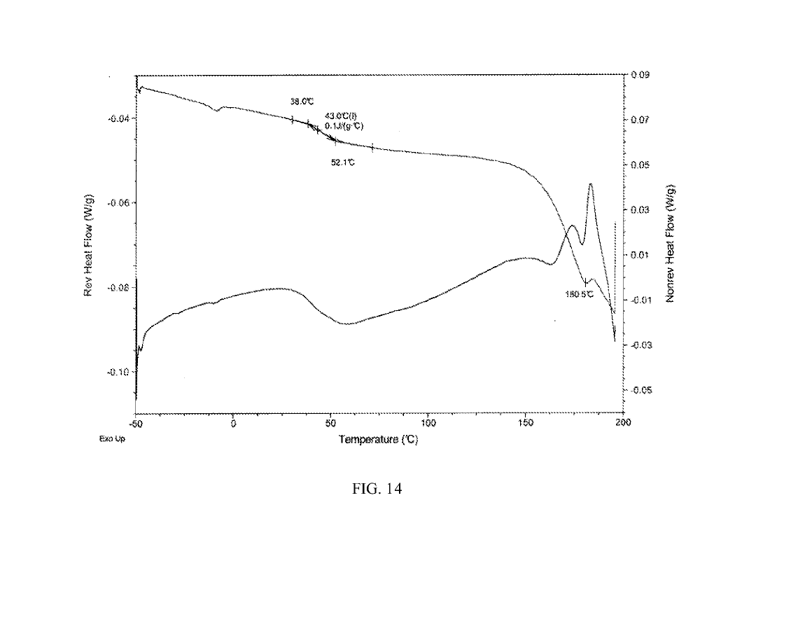

In FIG. 14 are shown the curves of a modulated DSC thermogram of an EC145 solid dispersion prepared in 50 mM citrate buffer/pH 6.2 containing 1.4 mg/mL EC145 and 10% glycine.

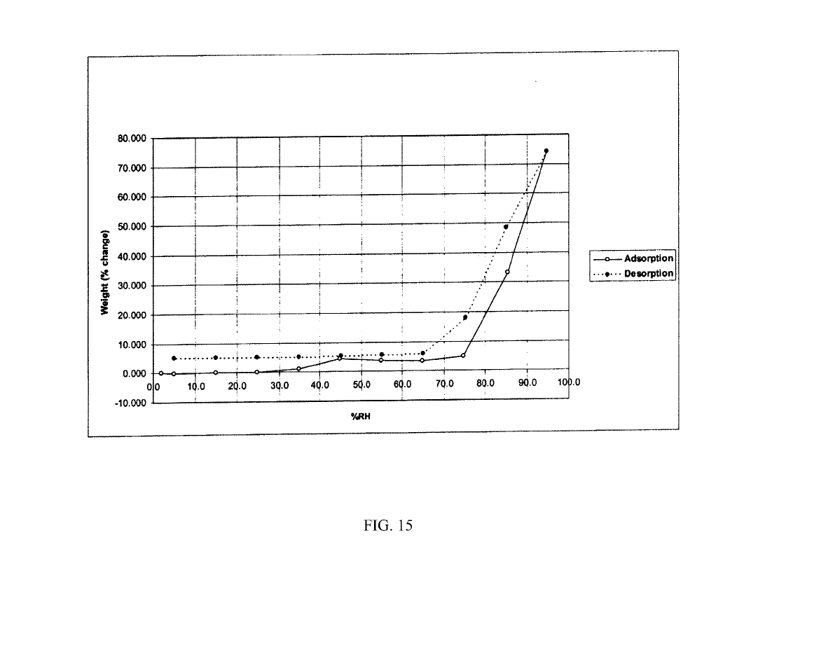

In FIG. 15 is shown the dynamic vapor sorption/desorption isotherm of an EC145 solid dispersion prepared in 50 mM citrate buffer/pH 6.2 containing 1.4 mg/mL EC145 and 10% glycine.

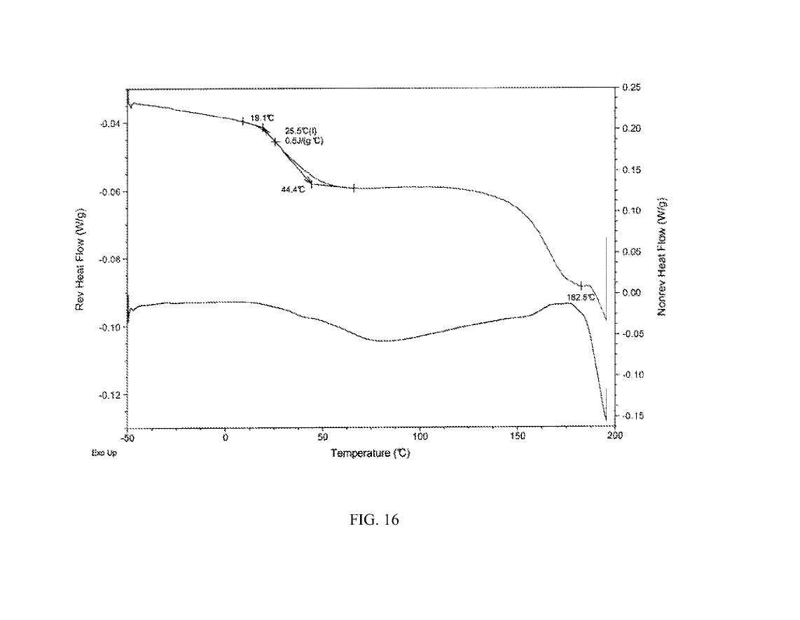

In FIG. 16 are shown the curves of a modulated DSC thermogram of an EC145 solid dispersion prepared in 50 mM citrate buffer/pH 6.2 containing 5.1 mg/mL EC145 and 3% glycine.

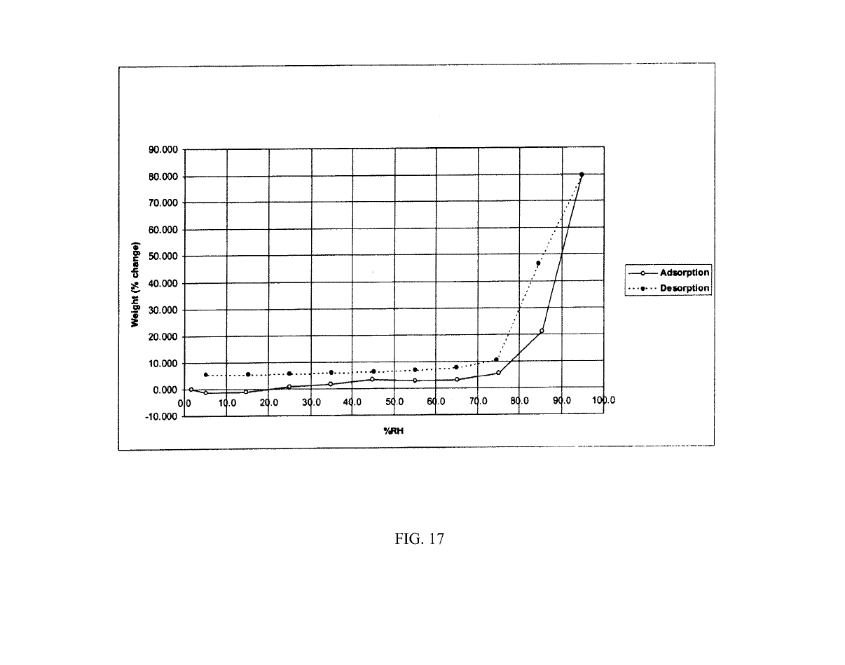

In FIG. 17 is shown the dynamic vapor sorption/desorption isotherm of an EC145 solid dispersion prepared in 50 mM citrate buffer/pH 6.2 containing 1.4 mg/mL EC145 and 3% mannitol.

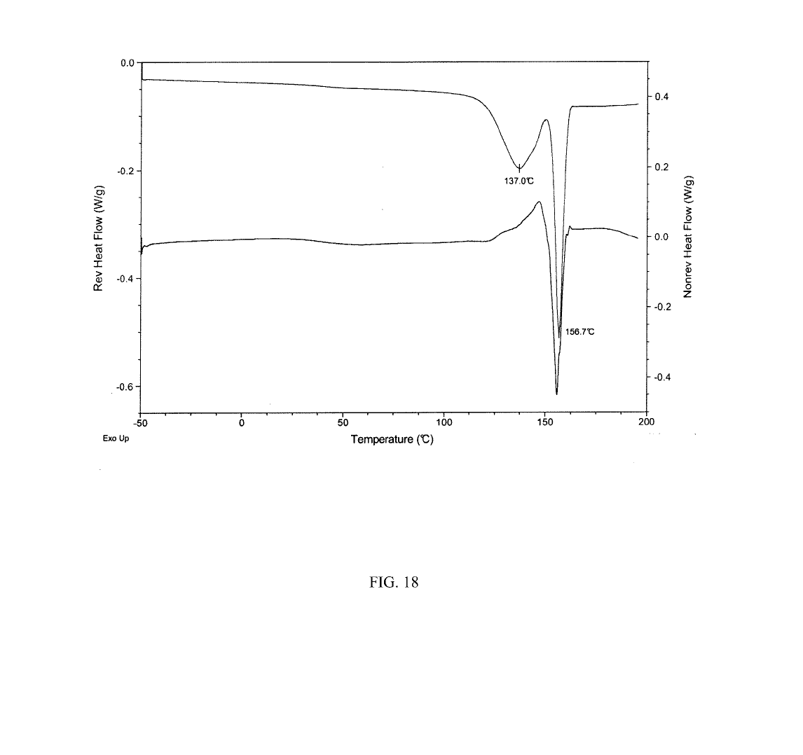

In FIG. 18 are shown the curves of a modulated DSC thermogram of an EC145 solid dispersion prepared in 50 mM citrate buffer/pH 6.2 containing 1.4 mg/mL EC145 and 10% mannitol.

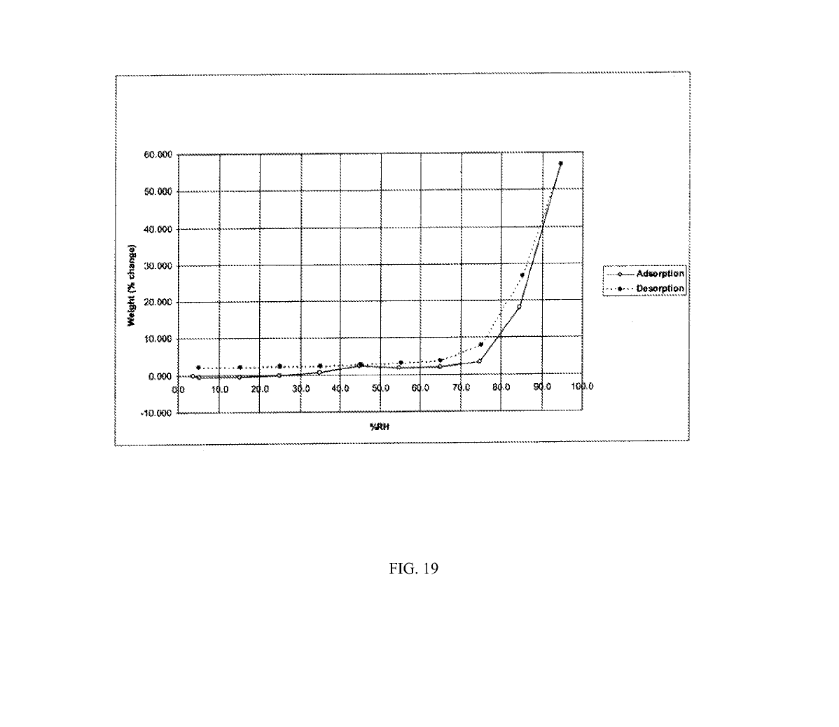

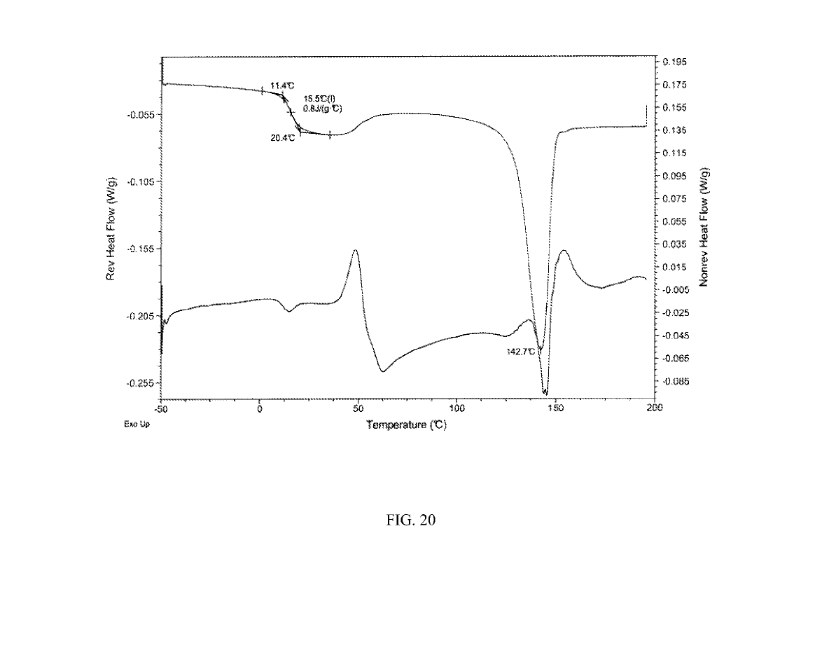

In FIG. 19 is shown the dynamic vapor sorption/desorption isotherm of an EC145 solid dispersion prepared in 50 mM citrate buffer/pH 6.2 containing 1.4 mg/mL EC145 and 10% mannitol. In FIG. 20 are shown the curves of a modulated DSC thermogram of an EC145 solid dispersion prepared in 50 mM citrate buffer/pH 6.2 containing 5.2 mg/mL EC145 and 3% mannitol.

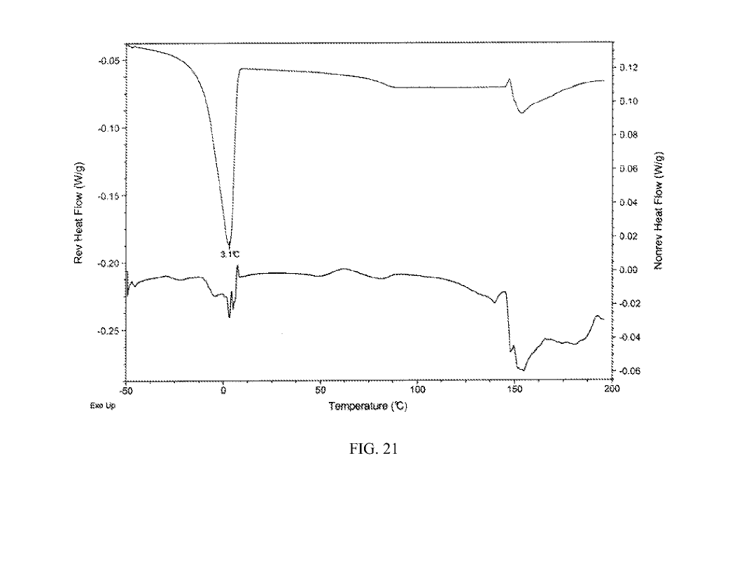

In FIG. 21 are shown the curves of a modulated DSC thermogram of an EC145 solid dispersion prepared in 50 mM citrate buffer/pH 6.2 containing 5.3 mg/mL EC145 and 3% PEG400.

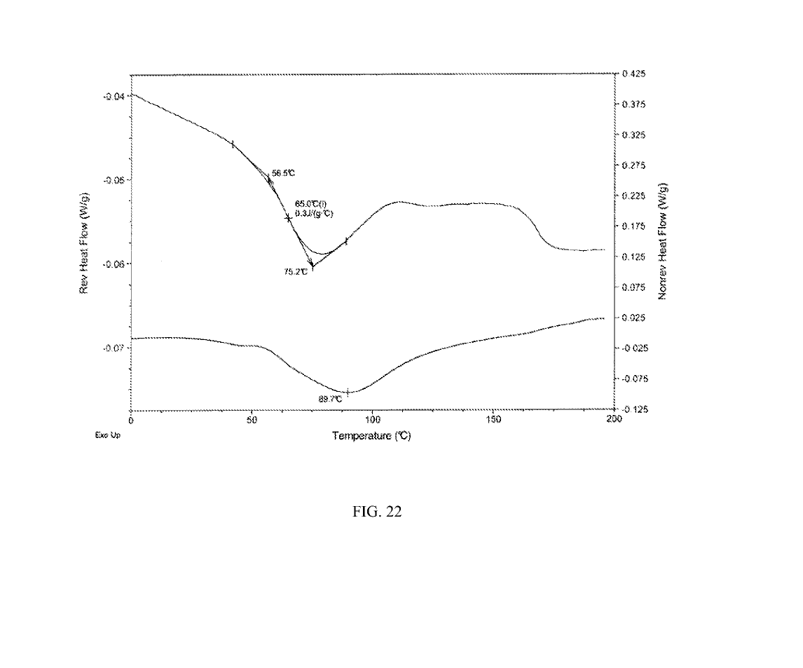

In FIG. 22 are shown the curves of a modulated DSC thermogram of an EC145 solid dispersion prepared in 50 mM citrate buffer/pH 6.2, containing 1.4 mg/mL EC145 and 3% PVP10.

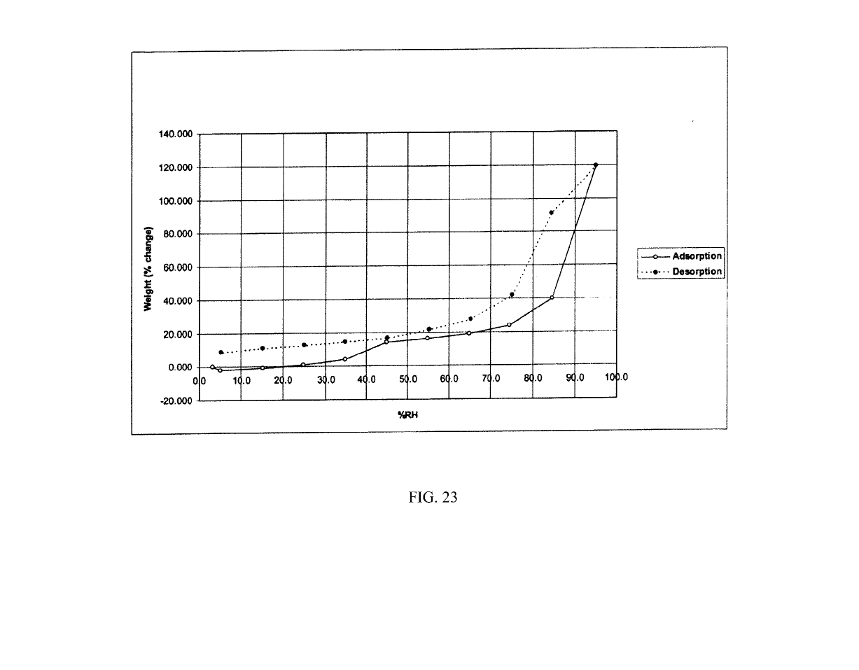

In FIG. 23 is shown the dynamic vapor sorption/desorption isotherm of an EC145 solid dispersion prepared in 50 mM citrate buffer/pH 6.2, containing 1.4 mg/mL EC145 and 3% PVP10.

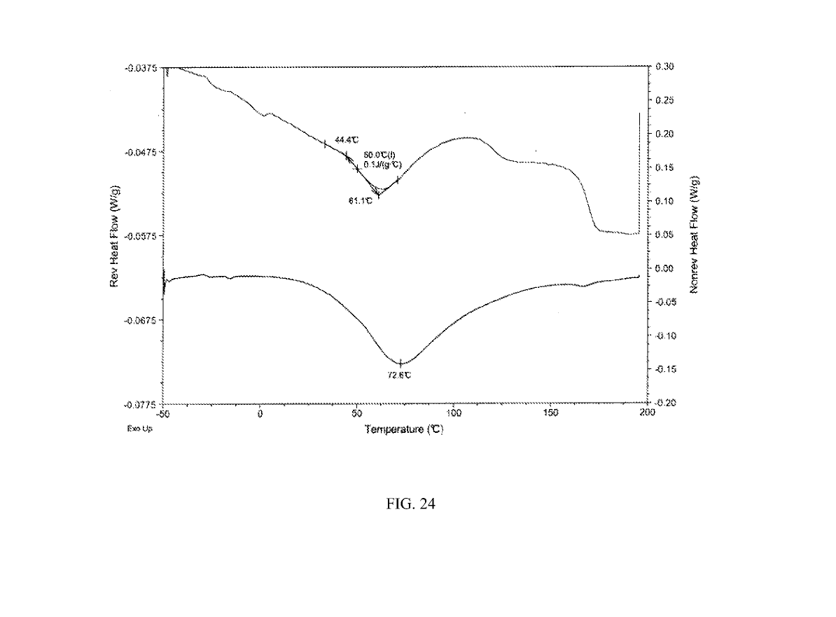

In FIG. 24 is shown the dynamic vapor sorption/desorption isotherm of an EC145 solid dispersion prepared in 50 mM citrate buffer/pH 6.2, containing 1.4 mg/mL EC145 and 3% PVP10.

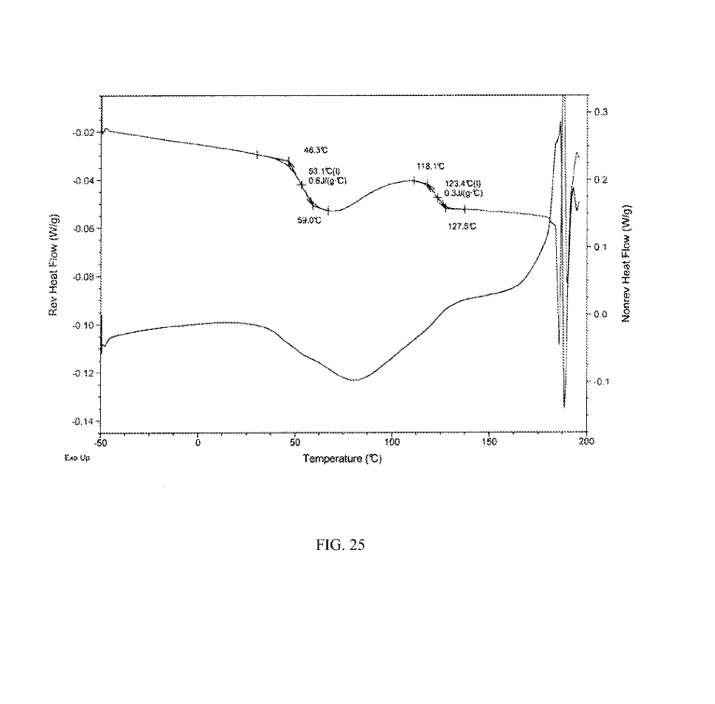

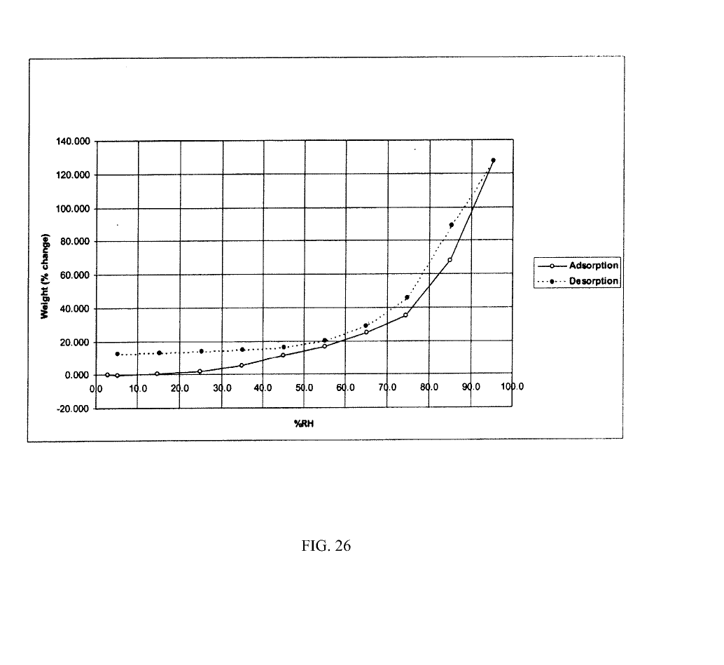

In FIG. 25 are shown the curves of a DSC thermogram of an EC145 solid dispersion prepared in 50 mM citrate buffer/pH 6.2, containing 1.4 mg/mL EC145 and 3% sucrose. In FIG. 26 is shown the dynamic vapor sorption/desorption isotherm of an EC145 solid dispersion prepared in 50 mM citrate buffer/pH 6.2, containing 1.4 mg/mL EC145 and 3% sucrose.

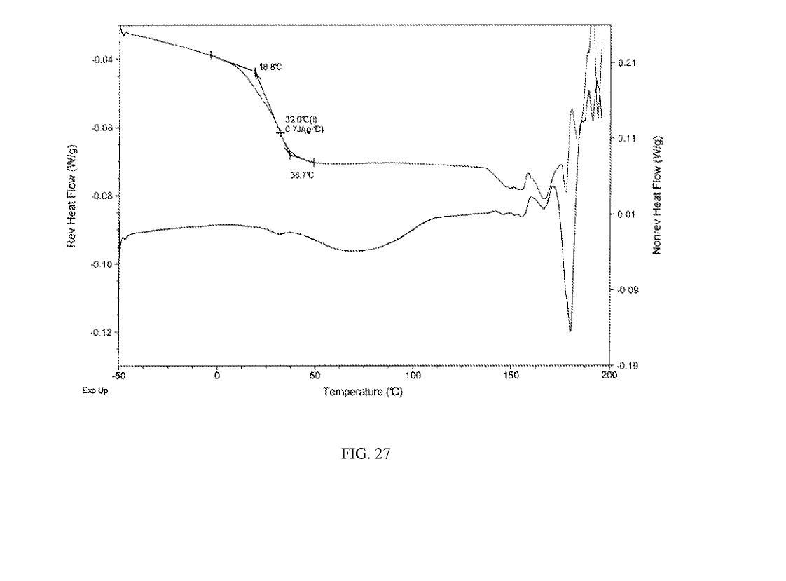

In FIG. 27 are shown the curves of a DSC thermogram of an EC145 solid dispersion prepared in 50 mM citrate buffer/pH 6.2, containing 1.4 mg/mL EC145 and 20% sucrose.

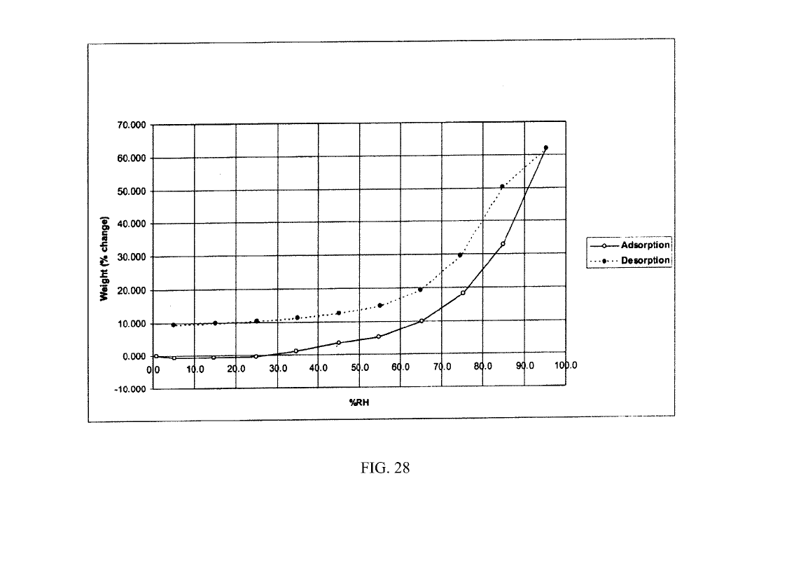

In FIG. 28 is shown the dynamic vapor sorption/desorption isotherm of an EC145 solid dispersion prepared in 50 mM citrate buffer/pH 6.2, containing 1.4 mg/mL EC145 and 20% sucrose.

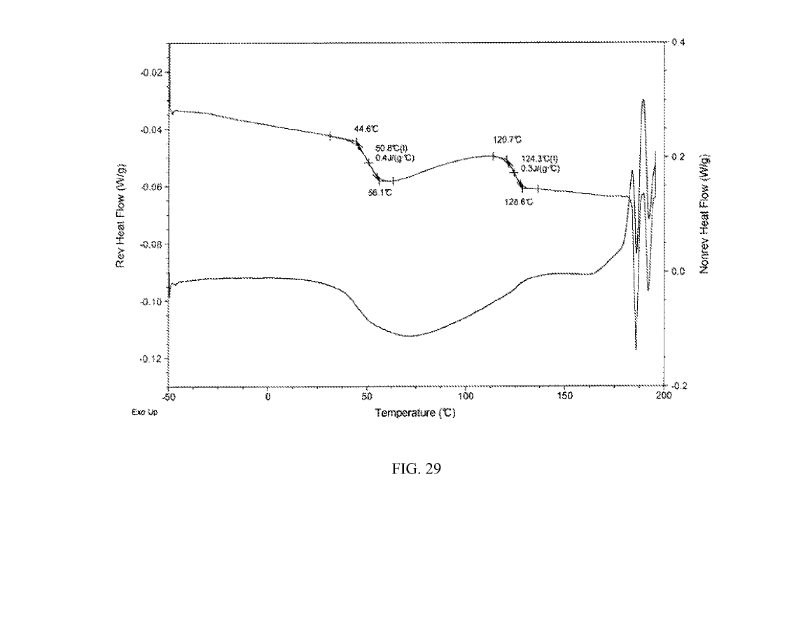

In FIG. 29 are shown the curves of a DSC thermogram of an EC145 solid dispersion prepared in 50 mM citrate buffer/pH 6.2, containing 5.2 mg/mL EC145 and 3% sucrose.

### DETAILED DESCRIPTION OF ILLUSTRATIVE EMBODIMENTS

One embodiment of the invention is a solid pharmaceutical composition comprising EC145 and a bulking agent. As noted above the term EC145 means the compound, or a pharmaceutically acceptable salt thereof; and the compound may be present in an ionized form, including a protonated form. It will be appreciated that the pH of a solution of EC145 may be adjusted, for example by the use of 1.0 N hydrochloric acid or 1.0 N sodium hydroxide solution, and removal of water from the solution will afford a corresponding pharmaceutically acceptable salt.

In the descriptions herein, according to the context, the components of a solid pharmaceutical composition including EC145 may be provided in dry weights or relative dry weights on a weight to weight (w/w) basis. Alternatively, the components of a solution which provides the solid pharmaceutical composition upon removal of the water and which solid provides a corresponding aqueous pharmaceutical composition upon reconstitution using an aqueous diluent, may be described, for example, in terms of molar concentration or percent (%) concentration. In general for the descriptions herein, pH is as measured on dilution of the solid composition with water for injection to afford EC145 at a calculated concentration of 1.4 mg/mL; and the bulking agent is % weight/volume at that concentration.

The bulking agent, also known as a stabilizing agent, may be any acceptable bulking agent. For the pharmaceutical compositions described herein, in one embodiment the bulking agent comprises dextrose, glucose, glycine, inositol, mannitol, sorbitol, sucrose, a polyethyleneglycol (PEG) such as PEG400, or a polyvinylpyrrolidine (PVP) such as PVP10, or a combination thereof in an individual or combined range of about 3% to about 20% and/or arginine or proline in an individual or combined range of about 0.1 M to about 0.5 M. For the pharmaceutical compositions described herein, in one embodiment the bulking agent comprises dextrose, inositol, mannitol, sorbitol or sucrose, or a combination thereof in an individual or combined range of about 3% to about 6% and/or arginine or proline in an individual or combined range of about 0.1 M to about 0.5 M. In another embodiment, the bulking agent comprises about 3% to about 10% glycine or mannitol. In another embodiment, the bulking agent comprises about 3% to about 4% mannitol and 0% to about 1% sucrose. In another embodiment, the bulking agent comprises about 3% mannitol. In another embodiment, the bulking agent can be used to make a lyophilized composition together with EC145.

Another embodiment of the solid pharmaceutical compositions described herein is an embodiment comprising a further excipient.

In one embodiment the further excipient comprises a buffer. In one embodiment, the pH of the buffer is about 5.0 to about 8.0. In another embodiment, the pH of the buffer is about 5.7 to about 6.6. In another embodiment, the pH of the buffer is about 6.0 to about 6.6. In another embodiment, the pH of the buffer is about 6.2±0.2.

The buffer may be any acceptable buffer known in the pharmaceutical arts for the indicated pH range and physiological compatibility. In addition a buffer may additionally act as a stabilizer, for example, as an antioxidant which does not reduce a disulfide bond. In one embodiment, the buffer comprises an ascorbate, sorbate, formate, lactate, fumarate, tartrate, glutamate, acetate, citrate, gluconate, histidine, malate, phosphate or succinate buffer. In another embodiment, the buffer comprises an ascorbate, lactate, tartrate, citrate, gluconate, malate, isocitrate or 2-hydroxybutyrate buffer.

In one embodiment, the concentration of the above buffer is about 20 mM to 150 mM. In one embodiment, the buffer comprises a citrate buffer. In one embodiment, the concentration of the above buffer is about 100 mM or is 100 mM. In another embodiment, the concentration of the above buffer is about 50 mM or is 50 mM. A further embodiment of a buffer is one wherein the buffer is a pH 6.2 citrate buffer.

In one embodiment, the solid pharmaceutical composition is one wherein the solid corresponds to about 27 parts trisodium citrate dihydrate, about 1.5 parts citric acid, and about 40-80 parts mannitol to 2.8 parts EC145 by weight. In another embodiment, the solid pharmaceutical composition is one wherein the solid corresponds to about 27 parts trisodium citrate dihydrate, about 1.5 parts citric acid, and about 60 parts mannitol to 2.8 parts EC145 by weight.

One embodiment of the composition for any of the embodiments of a solid pharmaceutical composition described herein is one wherein the solid is a lyophilized solid pharmaceutical composition.

One embodiment of the composition for any of the embodiments of a solid pharmaceutical composition described herein is one wherein the EC145 is X-ray amorphous.

One embodiment of the composition for any of the embodiments of a solid pharmaceutical composition described herein is one wherein the Raman spectrum of the solid comprises substantially the same spectrum as shown in FIG. 3 including a peak at about 1606 cm⁻¹.

One embodiment of the composition for any of the embodiments of a solid pharmaceutical composition described herein is one wherein the X-ray powder diffraction pattern of the solid comprises substantially the same pattern as shown in FIG. 2.

The above described compositions may each be obtained as a solid dispersion and further characterized by dynamic vapor sorption.desorption, as described below and shown in the drawings. In one embodiment of any of the above compositions, the composition is a solid dispersion wherein the % weight increase at 65% relative humidity in dynamic vapor sorption.desorption does not exceed about 20% or 20%. In another embodiment the % weight increase at 65% relative humidity in dynamic vapor sorption.desorption does not exceed about 10% or 10%. In another embodiment the % weight increase at 65% relative humidity in dynamic vapor sorption.desorption does not exceed about 5% or 5%.

The above described compositions may each be obtained as a solid dispersion and further characterized on a weight to weight dry basis, exclusive of residual water. In one embodiment, the solid components correspond to about 5-10 parts EC145, about 75-90 parts of a buffer and about 150-750 parts of a bulking agent. In another embodiment, the solid components correspond to about 5-10 parts EC145, about 75-90 parts of a citrate buffer and about 150-750 parts of a bulking agent, such as mannitol. In one embodiment, the solid components correspond to about 8.6 parts EC145, about 81 parts trisodium citrate dihydrate, about 4.6-4.8 parts citric acid, and about 180 parts glycine. In one embodiment, the solid components correspond to about 8.6 parts EC145, about 81 parts trisodium citrate dihydrate, about 4.6-4.8 parts citric acid, and about 600 parts glycine. In one embodiment, the solid components correspond to about 8.6 parts EC145, about 81 parts trisodium citrate dihydrate, about 4.6-4.8 parts citric acid, and about 180 parts mannitol. In one embodiment, the solid components correspond to about 8.6 parts EC145, about 81 parts trisodium citrate dihydrate, about 4.6-4.8 parts citric acid, and about 600 parts mannitol.

For a solid dispersion as described above, in one embodiment the residual water content is about 1.5 to about 5% by weight.

As another embodiment of the invention, there is described a method of producing a lyophilized solid pharmaceutical composition comprising EC145 and a bulking agent, and optionally further comprising a buffer, as described in any of the embodiments herein, comprising lyophilizing an aqueous solution of EC145 and a bulking agent, and wherein the solution optionally further comprises a buffer. In the Examples section, specific exemplification of such a process is provided.

In one embodiment there is described a method of producing a lyophilized pharmaceutical composition comprising amorphous EC145, which method comprises one or more of the steps (i) and (ii):
(i) completely freezing a liquid composition comprising EC145, a bulking agent as described in any of the embodiments herein, and optionally a buffer as described in any of the embodiments herein and an aqueous solvent at or below −20° C. prior to a primary drying step; and
(ii) the initial step of a primary drying stage comprising applying a vacuum to reduce the pressure effective to remove aqueous solvent from the frozen mixture of a liquid composition comprising EC145, a bulking agent as described in any of the embodiments herein, and optionally a buffer as described in any of the embodiments herein and an aqueous solvent, wherein the temperature is maintained at about −50° C. or below.

In another embodiment, there is described a method of producing a lyophilized pharmaceutical composition comprising X-ray amorphous EC145, which method comprises the steps of: (a) providing a liquid composition comprising EC145, a bulking agent, and, optionally, a buffer as described in any of the embodiments herein and an aqueous solvent in a container for the lyophilized product; (b) chilling the composition to a temperature of about −20° C. to about −50° C.; (c) freezing the composition to a temperature of about −20° C. to about −50° C., wherein the temperature is maintained for a sufficient time to provide a frozen mixture; (d) subjecting the frozen mixture to a primary drying stage, which comprises applying a vacuum to reduce the pressure effective to remove aqueous solvent from the frozen mixture, wherein the temperature is maintained at about −50° C. for a sufficient time for the first step of the primary drying and, while applying the vacuum, changing the temperature of the frozen mixture to a primary drying temperature, wherein the primary drying temperature is about −37° C., maintained for a sufficient time to complete the primary drying; (e) subjecting the composition to a secondary drying stage, which comprises applying a vacuum to reduce the pressure effective to remove aqueous solvent from the composition, and, while applying the vacuum, changing the temperature of the composition to a secondary drying temperature, wherein the secondary drying temperature is about 20° C., and maintaining the secondary drying temperature for a sufficient time to complete the secondary drying; (f) backfilling the vacuum chamber with an inert gas; and (g) sealing the container for the lyophilized product.

As an embodiment of the invention, there is described a lyophilized solid pharmaceutical composition comprising EC145 which is made by a process comprising lyophilizing a liquid composition comprising EC145, a bulking agent, optionally a buffer and an aqueous solvent. As another embodiment, there is described the above composition which is made by a process comprising one or more of the steps (i) and (ii):
(i) completely freezing the liquid composition comprising EC145, a bulking agent, an aqueous solvent and optionally a buffer at or below −20° C. prior to a primary drying step; and
(ii) the initial step of a primary drying stage comprising applying a vacuum to reduce the pressure effective to remove aqueous solvent from the frozen mixture of the liquid composition comprising EC145, a bulking agent, an aqueous solvent and optionally a buffer, wherein the temperature is maintained at about −50° C. for the first step of the primary drying.

For any of the above, an embodiment is the composition wherein the bulking agent comprises dextrose, glucose, glycine, inositol, mannitol, sorbitol, sucrose, a polyethyleneglycol (PEG), or a polyvinylpyrrolidine (PVP), or a combination thereof in an individual or combined range of about 3% to about 20% and/or arginine or proline in an individual or combined range of about 0.1 M to about 0.5 M. For any of the above, an embodiment is the composition wherein the bulking agent comprises dextrose, inositol, mannitol, sorbitol or sucrose, or a combination thereof, in an individual or combined range of about 3% to about 6% and/or arginine or proline in an individual or combined range of about 0.1 M to about 0.5 M. For any of the above, an embodiment is the composition wherein the bulking agent comprises about 3% to about 10% glycine or mannitol. For any of the above, an embodiment is the composition wherein the bulking agent comprises about 3% to about 4% mannitol and 0% to about 1% sucrose. For any of the above, an embodiment is the composition wherein the bulking agent comprises about 3% mannitol.

For any of the above, another embodiment is the composition which comprises a buffer wherein the pH of the buffer is about 5.0 to about 8.0. For any of the above, another embodiment is the composition which comprises a buffer wherein the pH of the buffer is about 5.7 to about 6.6. For any of the above, another embodiment is the composition which comprises a buffer wherein the pH of the pH of the buffer is about 6.0 to about 6.6. Another embodiment of the above is the composition wherein the pH of the buffer is about 6.2±0.2.

For any of the above, an embodiment is the composition wherein the EC145 is X-ray amorphous. For any of the above, an embodiment is the composition wherein the Raman spectrum of the solid comprises substantially the same spectrum as shown in FIG. 3 including a peak at about 1606 cm⁻¹. For any of the above, an embodiment is the composition wherein the X-ray powder diffraction pattern of the solid comprises substantially the same pattern as shown in FIG. 2.

In one embodiment of the invention, there is described a drug product comprising a solid pharmaceutical composition comprising EC145 as described in any of the embodiments herein. In one embodiment the drug product further comprises an ampoule or a sealed vial. In one embodiment the drug product further comprises a sealed vial. An embodiment for any of the drug products is one wherein the pharmaceutical composition comprises a citrate buffer. In one embodiment of the above, the drug product is a multidose form. In another embodiment of the above, the drug product is a single dose form (i.e., a unit dose form or a dosage unit). One embodiment of the above dosage unit is one which provides on dilution or reconstitution with an aqueous diluent a solution comprising EC145 for intravenous administration as 2.0 mL of an aqueous sterile liquid formulation, which dosage unit contains 1.4 mg/mL of EC145.

For any of the above embodiments, one embodiment is one wherein the drug product is able to maintain a purity specification for EC145 of greater than or equal to 94% over the course of a year at ambient temperature (25° C.±2° C.).

A further embodiment is a pharmaceutical composition obtained by reconstitution of a solid comprising EC145 and a bulking agent as described in any of the above embodiments. One embodiment is the above composition which composition comprises EC145 at a concentration of 1.4 mg/mL in an aqueous sterile liquid formulation the components of which comprise pH 6.2 citrate buffer, mannitol and water for injection.

An embodiment of the invention is a method of for treating a patient with a tumor bearing functionally active folate receptors comprising at least one of the steps of:
(a) dissolving the solid pharmaceutical composition described in any of the above embodiments in a pharmaceutically acceptable solvent to produce a pharmaceutically acceptable solution, and
(b) administering the solution to the patient in need thereof.
   One embodiment of the above is one wherein the tumor is an ovarian tumor or a lung tumor. One embodiment of the above is one wherein the tumor is an ovarian tumor. One embodiment of the above is one wherein the tumor is a platinum-resistant ovarian tumor. For any of the above methods, one embodiment is one wherein the patient is further treated with pegylated liposomal doxorubicin or with doxorubicin which is not of the pegylated liposomal form. In one embodiment of the above, treating comprises reducing cellular proliferation of malignant cells in a patient.

As used herein, a solid dispersion means a solid obtained by an evaporative process from a solution or suspension. In one embodiment, the dispersion is obtained from an aqueous solution. In one embodiment, the evaporative process is lyophilization.

As used herein, As used herein, adequate stability at ambient temperature means the EC145 is able to maintain a purity specification of greater than or equal to 94% over the course of a year at ambient temperature (25° C.±2° C.).

The term “amorphous” in the pharmaceutical arts is often meant to describe a material which is completely randomly oriented in the solid phase. That definition is overly restrictive with respect to the invention described herein. As used herein, the term “amorphous” means, for example, when applied to the API or excipient components of a composition to be analyzed according to the methods of the invention, that the X-ray powder diffraction pattern of such a component would yield a “halo” often referred to as an “amorphous halo” as that term is generally used by those of ordinary skill in the X-ray powder diffraction arts. Such a halo is significantly wider than peaks found in a pattern of a crystalline compound and indeed, may take up the majority of the angles scanned. Such a halo may be indicative of a completely randomly oriented solid, but it may also be indicative of a nanocrystalline material or other disordered solid which does have some degree of order, but on a smaller scale than that of a crystalline material. As used herein, the term “X-ray amorphous” means a material whose diffraction pattern exhibits one or more amorphous halos. As used herein, the terms “X-ray amorphous” and “amorphous” are synonymous unless otherwise specified.

For purposes of this specification and appended claims, unless otherwise indicated, all numbers expressing quantities, percentages or proportions, and other numerical values used in the specification and claims, are understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Any measured numerical value, however, inherently contains certain errors resulting from the standard deviation found in its particular testing measurements. It is to be understood that in the X-ray powder diffraction spectra the exact values measured for ° 2θ (or the corresponding d-spacings) may vary depending upon the particular sample analyzed and the particular analysis procedure used. A range of values of at least ±0.1 ° 2θ, and in some cases at least ±0.2 ° 2θ, may be typical. Measurements on independently prepared samples on different instruments may lead to a variability which is greater than ±0.1 ° 2θ and/or ±0.2 ° 2θ. It is to be understood that in Ramen spectra the exact values measured for wavelength or wavenumber (cm⁻¹) may vary depending upon the particular sample analyzed and the particular analysis procedure used. A range of values of at least ±1° cm⁻¹ may be typical for the same sample on different instruments. Lot to lot differences, particularly of differing formulations may be larger; thus measurements on independently prepared samples on different instruments may lead to variability which is greater than ±4° cm⁻¹, or in some cases greater than ±6° cm⁻¹, particularly for broad peaks.

Embodiments of the invention are further described by the following enumerated clauses:
1. A solid pharmaceutical composition comprising EC145 and a bulking agent.
2. The composition of clause 1 wherein:
   (a) the bulking agent comprises dextrose, glucose, glycine, inositol, mannitol, sorbitol, sucrose, a polyethyleneglycol (PEG), or a polyvinylpyrrolidine (PVP), or a combination thereof in an individual or combined range of about 3% to about 20% and/or arginine or proline in an individual or combined range of about 0.1 M to about 0.5 M; or
   (b) the bulking agent comprises dextrose, inositol, mannitol, sorbitol or sucrose, or a combination thereof, in an individual or combined range of about 3% to about 6% and/or arginine or proline in an individual or combined range of about 0.1 M to about 0.5 M; or
   (c) the bulking agent comprises about 3% to about 10% glycine or mannitol; or
   (d) the bulking agent comprises about 3% to about 4% mannitol and 0% to about 1% sucrose; or
   (e) the bulking agent comprises about 3% mannitol.
3. The composition of clause 1 or 2 comprising a further excipient.
4. The composition of clause 3 wherein the excipient comprises a buffer.
5. The composition of clause 4 wherein the buffer is an antioxidant which does not reduce a disulfide bond.
6. The composition of clause 4 or 5 wherein: (a) the pH of the buffer is about 5.0 to about 8.0; or (b) the pH of the buffer is about 5.7 to about 6.6; or (c) the pH of the buffer is about 6.0 to about 6.6; or (d) the pH of the buffer is about 6.2±0.2.
7. The composition of any of clauses 4-6 wherein: (a) the buffer comprises an ascorbate, sorbate, formate, lactate, fumarate, tartrate, glutamate, acetate, citrate, gluconate, histidine, malate, phosphate or succinate buffer; or (b) the buffer comprises an ascorbate, lactate, tartrate, citrate, gluconate, malate, isocitrate or 2-hydroxybutyrate buffer; or (c) the buffer comprises a citrate buffer.
8. The composition of any of clauses 4-7 wherein: (a) the concentration of the buffer is about 20 mM to 150 mM; or (b) the concentration of the buffer is about 100 mM or is 100 mM; or (c) the concentration of the buffer is about 50 mM or is 50 mM.
9. The composition of clause 4 wherein the buffer is a pH 6.2 citrate buffer.
10. The composition of clause 1 wherein:
   (a) the solid corresponds to about 27 parts trisodium citrate dihydrate, about 1.5 parts citric acid, and about 40-80 parts mannitol to 2.8 parts EC145 by weight; or
   (b) the solid corresponds to about 27 parts trisodium citrate dihydrate, about 1.5 parts citric acid, and about 60 parts mannitol to 2.8 parts EC145 by weight.
11. The composition any of clauses 1-10 wherein the solid is a lyophilized solid pharmaceutical composition.
12. The composition of any of clauses 1-11 wherein the EC145 is X-ray amorphous.
13. The composition of clause 1 wherein the Raman spectrum of the solid comprises substantially the same spectrum as shown in FIG. 3 including a peak at about 1606 cm⁻¹.
14. The composition of clause 1 wherein the X-ray powder diffraction pattern of the solid comprises substantially the same pattern as shown in FIG. 2.
15. The composition of clause 4 which is a solid dispersion wherein the % weight increase at 65% relative humidity in dynamic vapor sorption.desorption does not exceed: (a) about 20% or 20%, or (b) about 10% or 10%, or (c) about 5% or 5%.
16. The composition of clause 1 which is a solid dispersion wherein, on a weight to weight dry basis, exclusive of residual water, the solid components correspond to: (a) about 5-10 parts EC145, about 75-90 parts of a buffer and about 150-750 parts of a bulking agent; or (b) about 8.6 parts EC145, about 81 parts trisodium citrate dihydrate, about 4.6-4.8 parts citric acid, and about 180 parts glycine; or (c) about 8.6 parts EC145, about 81 parts trisodium citrate dihydrate, about 4.6-4.8 parts citric acid, and about 600 parts glycine; or (d) about 8.6 parts EC145, about 81 parts trisodium citrate dihydrate, about 4.6-4.8 parts citric acid, and about 180 parts mannitol; or (e) about 8.6 parts EC145, about 81 parts trisodium citrate dihydrate, about 4.6-4.8 parts citric acid, and about 600 parts mannitol.
17. The composition of clause 16 wherein the residual water content is about 1.5 to about 5% by weight.
18. A method of producing a lyophilized solid pharmaceutical composition comprising EC145 and a bulking agent, and optionally further comprising a buffer, as described in any of clauses 1-17, comprising lyophilizing an aqueous solution of EC145 and a bulking agent, wherein the solution optionally further comprises a buffer.
19. The method of clause 18, which method comprises one or more of the steps (i) and (ii):
   (i) completely freezing a liquid composition comprising EC145, a bulking agent as described in any of the embodiments herein, and optionally a buffer as described in any of the embodiments herein and an aqueous solvent at or below −20° C. prior to a primary drying step; and
   (ii) the initial step of a primary drying stage comprising applying a vacuum to reduce the pressure effective to remove aqueous solvent from the frozen mixture of a liquid composition comprising EC145, a bulking agent as described in any of the embodiments herein, and optionally a buffer as described in any of the embodiments herein and an aqueous solvent, wherein the temperature is maintained at about −50° C. or below.
20. A lyophilized solid pharmaceutical composition comprising EC145 which is made by a process comprising lyophilizing a liquid composition comprising EC145, a bulking agent, an aqueous solvent and optionally a buffer.
21. The composition of clause 20 which is made by a process comprising one or more of the steps (i) and (ii):
   (i) completely freezing the liquid composition comprising EC145, a bulking agent, an aqueous solvent and optionally a buffer at or below −20° C. prior to a primary drying step; and
   (ii) the initial step of a primary drying stage comprising applying a vacuum to reduce the pressure effective to remove aqueous solvent from the frozen mixture of the liquid composition comprising EC145, a bulking agent, an aqueous solvent and optionally a buffer, wherein the temperature is maintained at about −50° C. for the first step of the primary drying.
22. The composition of clause 20 or 21 wherein:
   (a) the bulking agent comprises dextrose, glucose, glycine, inositol, mannitol, sorbitol, sucrose, a polyethyleneglycol (PEG), or a polyvinylpyrrolidine (PVP), or a combination thereof in an individual or combined range of about 3% to about 20% and/or arginine or proline in an individual or combined range of about 0.1 M to about 0.5 M; or
   (b) the bulking agent comprises dextrose, inositol, mannitol, sorbitol or sucrose, or a combination thereof, in an individual or combined range of about 3% to about 6% and/or arginine or proline in an individual or combined range of about 0.1 M to about 0.5 M; or
   (c) the bulking agent comprises about 3% to about 10% glycine or mannitol; or
   (d) the bulking agent comprises about 3% to about 4% mannitol and 0% to about 1% sucrose; or
   (e) the bulking agent comprises about 3% mannitol.
23. The composition of any of clauses 20 to 22 which comprises a buffer wherein: (a) the pH of the buffer is about 5.0 to about 8.0; or (b) the pH of the buffer is about 5.7 to about 6.6; or (c) the pH of the buffer is about 6.0 to about 6.6; or (d) the pH of the buffer is about 6.2±0.2.
24. The composition of any of clauses 20 to 23 wherein the EC145 is X-ray amorphous.
25. The composition of clause 20 wherein the Raman spectrum of the solid comprises substantially the same spectrum as shown in FIG. 3 including a peak at about 1606 cm⁻¹.
26. The composition of clause 20 wherein the X-ray powder diffraction pattern of the solid comprises substantially the same pattern as shown in FIG. 2.
27. A drug product comprising a solid pharmaceutical composition comprising EC145 as described in any of clauses 1-17 or 20-26.
28. The drug product of clause 27 further comprising an ampoule or a sealed vial.
29. The drug product of clause 28 further comprising a sealed vial.
30. The drug product of clause 27 wherein the pharmaceutical composition comprises a citrate buffer.
31. The drug product of any of clauses 27-30 wherein the drug product is a multidose form.
32. The drug product of any of clauses 27-30 wherein the drug product is a single dose form.
33. The dosage unit of clause 32 which provides on dilution or reconstitution with an aqueous diluent a solution comprising EC145 for intravenous administration as 2.0 mL of an aqueous sterile liquid formulation, which dosage unit contains 1.4 mg/mL of EC145.
34. The drug product of any of clauses 27-33 wherein the EC145 is able to maintain a purity specification for EC145 of greater than or equal to 94% over the course of a year at ambient temperature (25° C.±2° C.).
35. A pharmaceutical composition obtained by reconstitution of a solid comprising EC145 and a bulking agent as described in any of clauses 1-17 or 20-26.
36. The composition of clause 35 which composition comprises EC145 at a concentration of 1.4 mg/mL in an aqueous sterile liquid formulation the components of which comprise pH 6.2 citrate buffer, mannitol and water for injection.
37. A method of treating a patient with a tumor bearing functionally active folate receptors comprising at least one of the steps of:
   (a) dissolving the solid pharmaceutical composition described in any of clauses 1-17 or 20-26 in a pharmaceutically acceptable solvent to produce a pharmaceutically acceptable solution, and
   (b) administering the solution to the patient in need thereof.
38. The method of clause 37, wherein the tumor is an ovarian tumor or a lung tumor.
39. The method of clause 38 wherein the tumor is an ovarian tumor.
40. The method of clause 39 wherein the tumor is a platinum-resistant ovarian tumor.
41. The method of any one of clauses 37-40 wherein the patient is further treated with pegylated liposomal doxorubicin or with doxorubicin which is not of the pegylated liposomal form.

The following examples further illustrate specific embodiments of the invention; however, the following illustrative examples should not be interpreted in any way to limit the invention.

### EXAMPLES

### Materials

EC145 API (active drug product) is prepared according the description of U.S. Pat. No. 7,601,332 or of WO 2011/014821.

Other materials, instruments and equipment are obtained from commercial sources, including the following: Water for injection (WFI); Trisodium Citrate Dihydrate, EMD 1.06432.0500; Citric Acid, J T Baker 0122-01; Mannitol, JT Baker 2553-01; Argon; Nitrogen; Filter, Pall 12122; Tubing; Vials, Wheaton # 223685/W008230, 5 mL, 20 mm, Tubing, Type I Glass; Stoppers, (West pharmaceutical #19700021 or 19700022) 20 mm, S-10-F451, 4432/50 Gray w/B2-40 coating (serum stopper); Crimps, Blue (with serum stopper); Stoppers, West 20 mm 4432/50, S-87-J, Gray w/B2-44 coating (split skirt lyophilization stopper); Crimps, Helvoet Pharma 110009704, Brown 6028 (with split skirt lyophilization stopper); Milli-Q water, Millipore Direct Q 3 UV System; Sodium phosphate monobasic monohydrate, Mallinckrodt 7868; Sodium phosphate dibasic dihydrate, Fisher S472-500; Sodium chloride, Mallinckrodt 7581; Potassium chloride, Fisher P330-500; Sodium Citrate Dihydrate, Aldrich 39,807-1; Sucrose, Sigma S3929-1KG; Sodium Hydroxide, JT Baker 3278-01; Hydrochloric Acid, EMD HX0603P-S; Glacial Acetic Acid, EMD AX0074-6; Triethylamine Acetate, Fisher, 04885-1; 5 N Ammonium Hydroxide, Acros, AC612570010; Acetonitrile, Sigma-Aldrich 34851-4L; HPLC column Waters Symmetry C18, 3.5 μm, 4.6×75 mm, P/N WAT066224; Guard column Waters Symmetry C18, 5 μm, 3.9×20 mm, P/N WAT054225.

### Instruments and Equipment

HPLC: Waters Alliance 2695 with Waters 2487 Dual λ Absorbance Detector; HPLC: Agilent 1200 with PDA detector; pH meter, pH-08, Corning 340; Autoclave, Hotpack Steam Sterilizer, PE5-004; Oven, VWR 1370FM; Oven, Gruenberg dry heat oven; Balance, Sartorius R300S; Balance, Sartorius CP34001; Pump, Watson Marlow 505S; Pipettor, Eppendorf Repeater Plus, with 50 mL Combitips; Lyophilizer: FTS LyoStar II with LyoManager II Data Collection; Capper, Westcapper NPW-500, 5A-018.

### Example

### Lyophilized EC145

Two vials of an aqueous solution of EC145 with a total volume of 22 mL were thawed at ambient temperature and transferred into four 20 mL lyophilization vials. The vials were transferred to a freezer for approximately 1.5 hours. Lyophilization was conducted using a LABCONCO freeze-drier which was pre-chilled to −72° C. (shelf temperature) before the samples were loaded. Samples were lyophilized for ˜30 minutes at −20° C., followed by another 38.5 hours at 20° C. The resulting fluffy pale yellow solid was combined into one vial for further characterization.

### Formulations

### Aqueous Formulations:

Provided below are formulations which may be used to provide EC145 at a concentration of 1.4 mg/mL of EC145. Single vials are used to provide a 2.5 mg bolus dose of EC145.

### Example

### pH 7.4, Phosphate-buffered EC145 Formulation

The following formulation provides a EC145 drug product (DP) for intravenous (IV) administration as 2.0 mL of an aqueous sterile liquid formulation, pH 7.4, in single-use clear glass vials with Flurotech™-coated rubber stoppers, which is stored frozen under inert gas. Each vial contains 1.4 mg/mL of EC145. The quantitative composition of the drug product is shown in the table below. Single vials are used to provide a 2.5 mg bolus dose of EC145. This formulation provides 10 mM phosphate buffer, pH 7.4; 138 mM sodium chloride, and 2.7 mM potassium chloride.

EC145 Drug Product Components

| | | | Amount per vial |
|---|---|---|---|
| | Function | Grade | (mg) |
| EC145 | Active | In-house | 2.8 |
| Sodium phosphate, | pH control | USP | 1.1 |
| monobasic monohydrate | tonicity | | |
| Disodium phosphate, | pH control | USP | 2.14 |
| dibasic dihydrate | Tonicity | | |
| Sodium chloride | Tonicity | USP | 16.12 |
| Potassium chloride | Tonicity | USP | 0.4 |
| Water for Injection | Solvent | WFI | QS to 2.0 mL |

### Example

### pH 6.2 Citrate-buffered EC145 Formulation

The following formulation provides a solution which is a 50 mM citrate buffered pH 6.2 EC145 solution.

EC145 Drug Product Components

| | | | Amount per vial |
|---|---|---|---|
| | Function | Grade | (mg) |
| EC145 | Active | In-house | 2.8 |
| Trisodium citrate | pH control | USP | 27 |
| dihydrate | tonicity | | |
| Citric acid | pH control | USP | 1.5 |
| | Tonicity | | |
| Water for Injection | Solvent | WFI | QS to 2.0 mL |

### Formulations for Lyophilization:

### Example

### pH 6.2 Citrate-buffered EC145 Formulation with 3% Mannitol

The following formulation provides a solution which is a pH 6.2 citrate-buffered EC145 solution containing 3% mannitol as bulking agent useful for lyophilization and reconstitution.

EC145 Drug Product Components

| | | | Amount per vial |
|---|---|---|---|
| | Function | Grade | (mg) |
| EC145 | Active | In-house | 2.8 |
| Trisodium citrate | pH control | USP | 27 |
| dihydrate | tonicity | | |
| Citric acid | pH control | USP | 1.5 |
| | Tonicity | | |
| Mannitol | Bulking agent, | USP | 60 |
| | Stabilizing Agent | | |
| | Tonicity | | |
| Water for Injection | Solvent | WFI | QS to 2.0 mL |

### Example

### pH 6.2 Citrate-buffered EC145 Formulation with 4% Mannitol/1% Sucrose

The same formulation as for 3% mannitol above, but with 80 mg mannitol and 20 mg sucrose.

### Example

### Placebo pH 6.2 Citrate-buffered Formulation with 3% Mannitol

The following formulation provides a placebo solution lacking which is a pH 6.2 citrate-buffered solution containing 3% mannitol as bulking agent useful for lyophilization and reconstitution.

Placebo Product Components

| | | | Amount per vial |
|---|---|---|---|
| | Function | Grade | (mg) |
| Trisodium citrate | pH control | USP | 27 |
| dihydrate | tonicity | | |
| Citric acid | pH control | USP | 1.5 |
| | Tonicity | | |
| Mannitol | Bulking agent, | USP | 60 |
| | Stabilizing Agent | | |
| | Tonicity | | |
| Water for Injection | Solvent | WFI | QS to 2.0 mL |

### Example

### Preparation of Lyophilized EC145 and Placebo Pharmaceutical Compositions

Lyophilization cycles were run with EC145 vials containing 3% mannitol, EC145 vials containing 4% mannitol/1% sucrose, and placebo vials (without EC145 API). Probes were placed within EC145 solution vials and placebo vials to record the solution temperature during the cycle. Before exposing the final product to air, all of the cycles were backfilled with Argon with the vials stoppered in the lyophilizer. Immediately after stoppering, the vials were crimped and labeled.

In a number of lyophilization runs of varying parameters, no visible differences could be seen between the vials that contained 3% mannitol and the vials that contained 4% mannitol/1% sucrose.

### Example

### Description of Formulation Process

A large flask is charged with excess WFI and sparged with inert gas for 30 minutes to reduce the oxygen content to <1.0 ppm. An in-process test is used to confirm the oxygen content before formulation is started. A constant, positive pressure inert gas blanket is maintained on the formulation solution throughout the formulation process.

Frozen EC145 drug substance (API) solution is removed from a freezer and thawed in a 20° C.-25° C. controlled temperature circulating water bath. The thawed API solution is added to a tared, inert gas purged vessel to determine the amount of API solution to be formulated. Based on the density and the EC145 concentration in the solution, the weight of solution added to the tared vessel is used to define the total final solution available for filling at 1.4 mg EC145/mL.

A vessel with stir bar is weighed and charged with 62.5% of the total volume of the final fill volume of WFI. Mannitol is added to provide a final concentration of 3% mannitol. Sodium citrate is added to the vessel followed by a rinse with sparged WFI. Citric acid is added to the vessel followed by a rinse with sparged WFI. The sparged solution is mixed until all the citric acid was dissolved.

A pH meter is standardized with pH 4 and 7 buffer standards to measure the pH of the solution. If the pH is not 6.0-6.2, then the pH is adjusted with 1.0M citric acid or 1.0M sodium citrate.

The vessel is wrapped in foil to shield the EC145 from light. The EC145 drug substance solution is added to the formulation vessel with stirring and sparging with inert gas. The drug substance containing vessel is rinsed twice with WFI solution. The mixture is stirred with sparging until a visually homogeneous mixture is obtained. The final target formulation weight is determined and the solution is charged with WFI to the target weight.

The solution is filtered through a 0.22 micron sterile filter, pre-wetted and bubble-point tested, using a peristaltic pump. An inert gas purge of the receiving vessel is maintained throughout the filtration process. Post filtration, the bubble point test is repeated to ensure that effective filtration was maintained throughout the process.

A fill head is calibrated to deliver 2.03 grams (2.0 mL, 2.8 mg EC145) of EC145 formulation solution to each vial. The fill amount is checked routinely during the fill process.

Stoppers are seated half-way on vials through the filling process.

Thermocouples are placed in appropriate vials in lyophilization trays, and the trays are lyophilized as per the cycle defined.

### Example

### Lyophilization Cycle

The following lyophilization cycle, using the pH 6.2 citrate-buffered EC145 solution containing 3% mannitol described above (2 mL in a 5 mL vial) provides the lyophilized EC145 formulation with a satisfactory cake appearance, which reconstitutes easily in water, and which retains a high API purity (>95%).

Vials are sparged with argon, filled with 2 mL of EC145 formulation, stoppered with a split skirt lyophilization stopper in the half seated position. As soon as a tray is filled and stoppered, it is placed in the lyophilizer at 5° C.
1. Pre-cool lyophilizer shelves to 5° C.
2. Load filled trays onto pre-cooled shelves.
3. Initiate lyophilization cycle after loading is complete.
4. Hold shelf temperature at 5° C. for 30-60 minutes

Alternatively, pre-cooling the shelves to −50° C. to 5° C. allows reduced time to precool the filled trays; and step 4 may not be needed.
5. Immediately ramp the shelf temperature to −50° C.
6. Hold the shelf temperature at −50° C. for 60 to 360 minutes.
7. Reduce the chamber pressure to 50 to 150 mTorr.
8. Hold the shelf temperature at −50° C. for 180 to 1740 minutes.
9. Ramp the shelf temperature to −37° C. over 73 minutes (0.20° C./minute).
10. Hold the shelf temperature at −37° C. for 60 to 720 minutes.
11. Ramp the shelf temperature to −20° C. over 150 minutes (0.11° C./minute).
12. Hold the shelf temperature at −20° C. for 60 to 360 minutes.
13. Ramp the shelf temperature to 20° C. to 30° C. over 60 to 360 minutes.
14. Hold the shelf temperature at 20° C. to 30° C. for 60 to 600 minutes.
15. Backfill the chamber with argon or nitrogen to 12.5 psia (870 mbar).
16. Collapse the shelves (fully stopper vials).

17. Backfill to atmospheric pressure with filtered air.
18. Unload lyophilizer.

A particular example of the above lyophilization cycle, with the initial cooling for 30 minutes at 5° C., may be represented in summary form as follows:

| Initial Freezing | 1 | 2 | 3 |
|---|---|---|---|
| Temp (° C.) | 5 | −50 | −50 |
| Time (min) | 30 | 0 | 360 |

| Primary and | | | | | | | |
|---|---|---|---|---|---|---|---|
| Secondary Drying | 1 | 2 | 3 | 4 | 5 | 6 | 7 |
| Temp (° C.) | −50 | −37 | −37 | −20 | −20 | 20 | 20 |
| Time (min) | 1740 | 73 | 720 | 150 | 360 | 360 | 600 |
| Vacuum (mTorr) | 100 | 100 | 100 | 100 | 100 | 100 | 100 |

The vials are then capped to provide the final product as a satisfactory cake in each vial.

### Example

### Characterization of Solid Formulation

### X-Ray Powder Diffraction (XRPD)

In FIG. 1 is shown the X-ray powder diffraction (XRPD) spectra of lyophilized EC145. In FIG. 2 is shown the X-ray powder diffraction pattern of a lyophilized, pH 6.2 citrate-buffered EC145 formulation with 3% mannitol.

XRPD patterns were collected with a PANalytical X'Pert PRO MPD diffractometer using an incident beam of Cu radiation produced using an Optix long, fine-focus source. An elliptically graded multilayer minor was used to focus Cu Kα a X-rays through the specimen and onto the detector. Prior to the analysis, a silicon specimen (NIST SRM 640d) was analyzed to verify the Si 111 peak position. A specimen of the sample was sandwiched between 3 μm thick films and analyzed in transmission geometry. A beam-stop, short antiscatter extension, and a helium atmosphere were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data-acquisition parameters for the pattern, including the divergence slit (DS) before the minor and the incident-beam antiscatter slit (SS), are as follows:

X-Ray Tube: Cu (1.54059 Å); Voltage: 45 kV; Amperage: 40 mA; Scan Range: 1.01-39.99 ° 2θ; Step Size: 0.017 ° 2θ; Collection Time: 1937 sec; Scan Speed: 1.2°/min; Slit: DS: 1/2°; SS: null; Revolution Time: 1.0 sec; Mode: Transmission; null short AS ext. used; He used.

### Raman Spectra

In FIG. 3 is shown the Raman spectrum of a lyophilized, pH 6.2 citrate-buffered EC145 formulation with 3% mannitol.

In FIG. 4 is shown the overlay of the Raman spectra of a lyophilized, pH 6.2 citrate-buffered EC145 formulation with 3% mannitol (top), a lyophilized placebo formulation (second from top), lyophilized EC145 (second from bottom), and a prior batch of lyophilized EC145 (bottom).

In FIG. 5 is shown the overlay of the expansion from 1500 to 1650 cm⁻¹ of the Raman spectra of a lyophilized, pH 6.2 citrate-buffered EC145 formulation with 3% mannitol (top), a lyophilized placebo formulation (second from top), lyophilized EC145 (second from bottom), and a prior batch of lyophilized EC145 (bottom).

Raman spectra were acquired on a FT-Raman module interfaced to a Nexus 670 FT-IR spectrophotometer (Thermo Nicolet) equipped with a germanium (Ge) detector. Wavelength verification was performed using sulfur and cyclohexane. Each sample was prepared for analysis by placing the sample into a pellet holder. Less than 1 W of Nd:YVO4 laser power (1064 nm excitation wavelength) was used to irradiate the sample. Each spectrum represents 1024 co-added scans collected at a spectral resolution of 4 cm-1.

### ¹³C CP/MAS NMR Spectroscopy

In FIG. 6 is shown the ¹³C CP/MAS NMR spectrum of lyophilized EC145, externally referenced to glycine at 176.5 ppm. In FIG. 7 is shown the ¹³C CP/MAS NMR spectrum of a lyophilized, pH 6.2 citrate-buffered EC145 formulation with 3% mannitol.

Solid-state 13C cross polarization magic angle spinning (CP/MAS) NMR spectra were acquired at ambient temperature on a Varian UNITYINOVA-400 spectrometer (Larmor frequencies: 13C=100.543 MHz, 1H=399.787 MHz). The samples were packed into 4 mm PENCIL type zirconia rotors and rotated at 12 kHz at the magic angle. The spectra were acquired with phase modulated (SPINAL-64) high power ¹H decoupling during the acquisition time using a ¹H pulse width of 2.5 μsed (90°), a ramped amplitude cross polarization contact time of 2 or 5 msec, a 30 or 50 msec acquisition time, a 5-300 second delay between scans, and a spectral width of 45 kHz. The free induction decay (FID) was processed using Varian VNMR 6.1C software with 65536 points and an exponential line broadening factor of 20 or 50 Hz to improve the signal-to-noise ratio. The first three data points of the FID were back predicted using the VNMR linear prediction algorithm to produce a flat baseline. The chemical shifts of the spectral peaks were externally referenced to the carbonyl carbon resonance of glycine at 176.5 ppm. The data acquisition and processing parameters for this spectrum are: Ambient temperature; Sequence: xpolvtlrhol; Rexa. delay: 5.000 sec; Acq. time: 0.030 sec; Spectral width: 44994.4 Hz (447.514 ppm); 1600 scans; Acquired points: 2700; Cross polarization contact time: 2 msec.

### Infrared Spectroscopy

In FIG. 8 is shown the infra red spectrum (DRIFTS) of a lyophilized, pH 6.2 citrate-buffered EC145 formulation with 3% mannitol.

IR spectra were acquired on a Magna-IR 560® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. Wavelength verification was performed using NIST SRM 1921b (polystyrene). A diffuse reflectance accessory (the Collector™, Thermo Spectra-Tech) was used for sampling. Sample preparation consisted of physically mixing the sample with KBr, placing the sample into a 13 mm diameter cup and leveling the material. Each spectrum represents 256 co-added scans collected at a spectral resolution of 4 cm-1. The background data set was acquired with KBr powder. A Log 1/R (R=reflectance) spectrum was obtained by taking a ratio of these two data sets against each other and then converting to Kubelka-Munk units.

### Thermal Analyses

In FIG. 9 are shown the DSC (left scale, left and lower curve) and TGA (right scale, upper and right curve) thermograms of a lyophilized, pH 6.2 citrate-buffered EC145 formulation with 3% mannitol.

DSC was performed using a TA Instruments Q2000 differential scanning calorimeter. Temperature calibration was performed using NIST traceable indium metal. The sample was placed into an aluminum DSC pan, covered with a lid, and the weight was accurately recorded. A weighed aluminum pan configured as the sample pan was placed on the reference side of the cell. For the data shown, the sample size was 2.0500 mg in a pan that was crimped at T₀; and the scan was run −30° C. to 250° C., at 10° C./min.

TG analyses were performed using a TA Instruments Q5000 IR thermogravimetric analyzer. Temperature calibration was performed using nickel and Alumel™. Each sample was placed in an aluminum pan. The sample was hermetically sealed, the lid pierced, then inserted into the TG furnace. The furnace was heated under nitrogen. For the data shown, the sample size was 2.7780 mg; and the scan was run 0° C. to 350° C., at 10° C./min.

### Example

### Stability Studies

Stability studies for an aqueous, pH 7.4 phosphate-buffered EC145 formulation (Liquid) refrigerated at 5° C. and for a lyophilized, pH 6.2 citrate-buffered EC145 formulation with 3% mannitol (Lyophilized, two development lots Lyo A and Lyo B, and a lot prepared as described in the above table Lyo C) at 25° C.±2° C. (as measured on reconstitution by dilution with water for injection) are provided in the following table.

| | Liquid | Lyo A | Lyo B | Lyo C |
|---|---|---|---|---|
| Time | Purity | Purity | Purity | Purity |
| (~mo) | (%) | Purity | Purity | Purity |
| 0 | 94.0 | 91.8 | 96.99 | 96.6 |
| 1 | | 89.5 | 96.46 | 97.7 |
| 2 | | | | 97.2 |
| 3 | 95.8 | 87.70 | 95.13 | 97.1 |
| 4 | | | | 97.0 |
| 5 | | | | 96.4 |
| 6 | 93.4 | 88.13 | 95.10 | 97.2 |
| 9 | 89.8 | 88.82 | 95.76 | 97.9 |
| 12 | | 85.59 | 93.83 | 96.7 |

### Example

### Solid Dispersion Screen of EC145

A solid dispersion screen of EC145 was conducted using six different excipients, including glucose, glycine, mannitol, PEG400, PVP10, and sucrose, at two different loadings (3% and 10 or 20% (w/v)) and EC145 at different concentrations as shown in the table below. Solid dispersion samples were generated by lyophilization in 50 mM citrate buffer, pH 6.2, according to the following lyophilization conditions.

| Temperature | Time |
|---|---|
| Samples were frozen in a −80° C. freezer | ~18 hours |
| −55° C. | 140 hours |
| −50° C. | 24 hours |
| −45° C. | 24 hours |
| −20° C. | 25.5 hours |
| 20° C. | 20.5 hours |

### Results

Visual Observation of the Lyophilized Product

| Analysis | | |
|---|---|---|
| Number | Sample Description | Observation* |
| 1 | 5.2 mg/mL EC145, 3% glucose | Pale yellow cake with smooth surface. |
| 2 | 5.1 mg/mL EC145, 3% glycine | Foamy yellow cake with some shrinkage |
| 3 | 5.2 mg/mL EC145, 3% mannitol | Pale yellow cake with smooth surface |
| 4 | 5.3 mg/mL EC145, 3% PEG400 | Shrunk yellow cake |
| | | (Solid was moist and attached to spatula tip) |
| 5 | 5.2 mg/mL EC145, 3% PVP10 | Pale yellow cake with smooth surface |
| 6 | 5.2 mg/mL EC145, 3% sucrose | Pale yellow cake with smooth surface |
| | Placebo: 3% glucose | White cake with smooth surface |
| | Placebo: 3% glycine | Small amount of powdery solid |
| | Placebo: 3% mannitol | White cake with smooth surface |
| | Placebo: 3% PEG400 | Small amount of white wet material on the bottom of the vial |
| | Placebo: 3% PVP10 | White cake with smooth surface |
| | Placebo: 3% sucrose | White cake with smooth surface |
| | 1.4 mg/mL EC145, 3% glucose | Pale yellow cake with smooth surface |
| 7 | 1.4 mg/mL EC145, 3% glucose | Pale yellow cake with smooth surface |
| | 1.4 mg/mL EC145, 3% glycine | Pale yellow powder |
| 8 | 1.4 mg/mL EC145, 3% glycine | Pale yellow powder |
| | 1.4 mg/mL EC145, 3% mannitol | Pale yellow cake with smooth surface |
| 9 | 1.4 mg/mL EC145, 3% mannitol | Pale yellow cake with smooth surface |
| | 1.4 mg/mL EC145, 3% PEG400 | Small amount of tacky yellow solid |
| | 1.4 mg/mL EC145, 3% PEG400 | Small amount of tacky yellow solid |
| | 1.4 mg/mL EC145, 3% PVP10 | Pale yellow cake with smooth surface |
| 10 | 1.4 mg/mL EC145, 3% PVP10 | Pale yellow cake with smooth surface |
| | 1.4 mg/mL EC145, 3% sucrose | Pale yellow cake with smooth surface |
| 11 | 1.4 mg/mL EC145, 3% sucrose | Pale yellow cake with smooth surface |
| | 1.4 mg/mL EC145, 20% glucose | Cake collapsed during secondary drying at 20° C. Resulted |
| | | in pale yellow foamy liquid. |
| 12 | 1.4 mg/mL EC145, 10% glycine | Pale yellow cake with smooth surface |
| 13 | 1.4 mg/mL EC145, 10% mannitol | Pale yellow cake with smooth surface |
| | 1.4 mg/mL EC145, 20% PEG400 | Melted at minus 20° C. Cloudy yellow solution |
| | 1.4 mg/mL EC145, 20% PVP10 | EC145 precipitated when added to 20% PVP solution |
| 14 | 1.4 mg/mL EC145, 20% sucrose | Pale yellow cake with smooth surface on top, a little foamy |
| | | on the bottom. |
| | | (Solid is dense. As broken with a spatula, a small piece of |
| | | tacky solid attached to the spatula tip. The rest of the solid |
| | | appeared dry and non-sticky. |

*When cakes were broken with a spatula tip, the solid appeared to be dry and fluffy unless specified in the table.

The samples indicated by Analysis Number were further analyzed for impurity profiles using a standard EC145 liquid chromatography assay.

The estimated compositions by weight, excluding residual water, of the solid dispersions in the above table are indicated in the following table In each case the solution was made up to a volume of 6 mL prior to lyophilization.

Estimated Dry Basis Composition of Lyophilized Product

| | | trisodium | | | |
|---|---|---|---|---|---|
| | EC145 | citrate | citric acid | excipient | |
| Sample Description | (mg) | dihydrate (mg) | (mg) | (mg) | |
| 5.2 mg/mL EC145, 3% glucose | 31.1 | 81.22 | 4.62 | 180 | glucose |
| 5.1 mg/mL EC145, 3% glycine | 30.8 | 81.25 | 4.64 | 180 | glycine |
| 5.2 mg/mL EC145, 3% mannitol | 31.1 | 81.12 | 4.69 | 180 | mannitol |
| 5.3 mg/mL EC145, 3% PEG400 | 31.7 | 81.37 | 4.66 | 180 | PEG 400 |
| 5.2 mg/mL EC145, 3% PVP10 | 30.9 | 81.38 | 4.70 | 180 | PVP |
| 5.2 mg/mL EC145, 3% sucrose | 31.2 | 81.17 | 4.66 | 180 | sucrose |
| Placebo: 3% glucose | 0 | 81.22 | 4.62 | 180 | glucose |
| Placebo: 3% glycine | 0 | 81.25 | 4.64 | 180 | glycine |
| Placebo: 3% mannitol | 0 | 81.12 | 4.69 | 180 | mannitol |
| Placebo: 3% PEG400 | 0 | 81.37 | 4.66 | 180 | PEG 400 |
| Placebo: 3% PVP10 | 0 | 81.38 | 4.70 | 180 | PVP |
| Placebo: 3% sucrose | 0 | 81.17 | 4.66 | 180 | sucrose |
| 1.4 mg/mL EC145, 3% glucose | 8.6# | 81.22 | 4.62 | 180 | glucose |
| 1.4 mg/mL EC145, 3% glucose | 8.6 | 81.22 | 4.62 | 180 | glucose |
| 1.4 mg/mL EC145, 3% glycine | 8.6 | 81.25 | 4.64 | 180 | glycine |
| 1.4 mg/mL EC145, 3% glycine | 8.6 | 81.25 | 4.64 | 180 | glycine |
| 1.4 mg/mL EC145, 3% mannitol | 8.6 | 81.12 | 4.69 | 180 | mannitol |
| 1.4 mg/mL EC145, 3% mannitol | 8.6 | 81.12 | 4.69 | 180 | mannitol |
| 1.4 mg/mL EC145, 3% PEG400 | 8.6 | 81.37 | 4.66 | 180 | PEG 400 |
| 1.4 mg/mL EC145, 3% PEG400 | 8.6 | 81.37 | 4.66 | 180 | PEG 400 |
| 1.4 mg/mL EC145, 3% PVP10 | 8.6 | 81.38 | 4.70 | 180 | PVP |
| 1.4 mg/mL EC145, 3% PVP10 | 8.6 | 81.38 | 4.70 | 180 | PVP |
| 1.4 mg/mL EC145, 3% sucrose | 8.6 | 81.17 | 4.66 | 180 | sucrose |
| 1.4 mg/mL EC145, 3% sucrose | 8.6 | 81.17 | 4.66 | 180 | sucrose |
| 1.4 mg/mL EC145, 20% glucose | 8.6 | 81.14 | 4.68 | 1200 | glucose |
| 1.4 mg/mL EC145, 10% glycine | 8.6 | 81.16 | 4.69 | 600 | glycine |
| 1.4 mg/mL EC145, 10% mannitol | 8.6 | 81.36 | 4.82 | 600 | mannitol |
| 1.4 mg/mL EC145, 20% PEG400 | 8.6 | 81.14 | 4.86 | 1200 | PEG 400 |
| 1.4 mg/mL EC145, 20% PVP10 | 8.6 | 81.22 | 4.78 | 1200 | PVP |
| 1.4 mg/mL EC145, 20% sucrose | 8.6 | 81.19 | 4.73 | 1200 | sucrose |

#60 μL of a 143 mg/mL EC145 solution is contained in the final 6 mL solution; dilution with the various buffer/excipient media to 6 mL affords a final EC145 concentration of 1.4 ml/mL.

### Physical Properties of the Lyophile

Physical property of each lyophile was observed visually. Most of the compositions formed cake with smooth surface, as shown in the above table.

Foamy cake was observed in formulations containing glycine and high concentration of EC145 at 5 mg/mL. Powder was observed at 1.4 mg/mL EC145 and the placebo sample without EC145. Formulations containing 3% PEG400 deliquesced during lyophilization. Formulation containing 20% PEG400 melted in the lyophilization chamber as temperature increased to −20° C. Formulation containing 20% glucose deliquesced (cake collapsed and resulted in foamy liquid) during the secondary drying at 20° C.

Solids with acceptable physical properties were obtained under the conditions of the solid dispersion screen from glucose, glycine, mannitol, PVP10, and sucrose. Deliquescence occurred in dispersions with PEG400.

### Solid State Characterization of the Lyophiles

Solid dispersions of EC145 were characterized using solid-state techniques, specifically high-quality, low background x-ray powder diffractometry (XRPD) and Fourier-transform infrared (FTIR) spectroscopy to provide a ‘finger print’ for the composition, differential scanning calorimetry (modulated) to determine the glass transition temperature (T_{g}), dynamic (water) vapor sorption analysis for hygroscopicity evaluation, hot state microscopy to observe physical changes (e.g., cake collapse temperature), and Karl Fischer titration to determine water content.

Solid dispersion samples generated from glucose, PVP10, and sucrose were determined to be X-ray amorphous, but those from glycine and mannitol contained crystalline material. All of the dispersions appeared to be very hygroscopic, indicated by significant weight changes during the vapor sorption/desorption analyses. Overall, solid prepared from lower excipient loading at 3% retained more water than those from higher excipient loadings at 10 or 20%.

Crystalline components were observed in dispersions containing glycine and mannitol. Because of the crystallization of glycine and mannitol during lyophilization, the glass transition temperature determined from the DSC thermogram is only from a small fraction of the amorphous material in the dispersion. The T_{g} value is not a valuable criterion for miscibility evaluation of the dispersion.

X-ray amorphous materials were obtained from dispersion containing glucose, PVP10, and sucrose. Because of the high hygroscopicity of EC145 and the excipients, the T_{g} value was significantly affected by water content. There was no correlation between T_{g} and EC145 loading in solids containing glucose and PVP10. In solid containing EC145 and 3% sucrose, two glass transition events, namely T_{g1} (51-53° C.) and T_{g2} (123-124° C.), were observed. However, a single T_{g} at a lower temperature (32-38° C.) was observed in the placebo sample and an EC145 dispersion containing 20% sucrose.

The IR spectra attributed to EC145 can not be identified clearly in all of the dispersions due to low EC145 content in the samples and overlapping of IR signals from EC145 and the excipients. Minor differences, possibly attributed to EC145 in the dispersion, were observed in the second derivative IR spectra at different EC145 loadings.

In the impurity profiles obtained for the samples indicated by Analysis Numbers 1-14, the total impurities for each of the samples was less than 4%.

### Detailed Characterizations

Solid Dispersions with Glucose

Four compositions were prepared with glucose: a placebo sample with 3% glucose, EC145 at 1.4 mg/mL in 3% and 20% glucose, and EC145 at 5.2 mg/mL in 3% glucose. Sample prepared in 20% glucose deliquesced during the secondary drying at 20° C., further characterization of it was not conducted.

The DSC thermogram of the placebo sample exhibited a single glass transition event at 30.6° C. that is presumably attributed to amorphous glucose.

Solid generated from 1.4 mg/mL EC145 in 3% glucose was determined to be X-ray amorphous. The XRPD pattern features two broad diffuse scattering maxima between 1 and 32 degree 2θ. A glass transition event was observed at 16.9° C. (FIG. 10). Similar phase transition was also observed at 23-33° C. by hot stage microscopy. This glass transition temperature is lower than that observed in the placebo sample. The low glass transition temperature in this sample may be a result of high water content (5.8% by Karl Fischer).

The DVS results suggested that the material is very hygroscopic. The material exhibited a negligible weight change upon equilibration at 5% RH. However, a significant weight gain of approximately 137% was observed during the sorption step with the greatest amount gained between 75 and 95% RH. Similarly, a significant weight loss of 126% was observed in the desorption step. Minor hysteresis was observed (FIG. 11). The sample deliquesced upon completion of the test.

The DSC thermogram of solid generated from 5.2 mg/mL EC145 in 3% glucose exhibited a glass transition event at 44.0° C. which is higher than that observed in the placebo and the sample with low EC145 loading. A higher glass transition temperature for high EC145 loading may be indicative of a potential interaction between EC145 and glucose. However, an inconsistent result was observed at low EC145 loading.

In summary, solid dispersion of EC145 in glucose is determined to be X-ray amorphous. The material is very hygroscopic. A weight change of 137% and 126% was observed in the sorption and desorption step, respectively. Low glass transition temperature (T_{g}, ˜17° C.) was observed in dispersion containing 1.4 mg/mL EC145 and a higher T_{g} at ˜44° C. in dispersion containing 5.2 mg/mL EC145. Different T_{g} values in the two dispersions might be a result of different EC145 concentration or different water content. Due to low EC145 content in the samples and the overlapping between the signals from EC145 and the excipient, the IR spectrum attributed to EC145 can not be identified clearly.

Solid Dispersions with Glycine

Four compositions were prepared with glycine: a placebo sample with 3% glycine, EC145 at 1.4 mg/mL in 3% and 10% glycine, and EC145 at 5.2 mg/mL in 3% glycine.

The DSC thermogram of the placebo sample exhibited a single glass transition event at 39.9° C. that is presumably attributed to amorphous glycine.

The XRPD pattern of solid generated from 1.4 mg/mL EC145 in 3% glycine exhibited some features indicative of crystalline phase and diffuse scattering. Diffuse scattering may be attributed to a separate amorphous phase, disordered/defective crystalline or nano-crystalline component.

The DSC thermogram of solid generated from 1.4 mg/mL EC145 in 3% glycine exhibited a glass transition event at 24.3° C. followed by a broad endotherm at 182.8° C. (FIG. 12), possibly due to melting of the crystalline component.

The DVS results suggested that the material is very hygroscopic. The material exhibited a negligible weight change upon equilibration at 5% RH. However, a significant weight gain of approximately 103% was observed during the sorption step with the greatest amount gained between 75 and 95% RH. Similarly, a significant weight loss of 95% was observed in the desorption step (FIG. 13). There was significant hysteresis from 95 to 65% RH.

Water content in the sample was determined to be 3.68% by Karl Fischer titration.

The material containing 1.4 mg/mL EC145/10% glycine is determined to be crystalline. The DSC thermogram exhibited a weak glass transition event at 43.0° C. followed by a broad endotherm at 180.5° C. (FIG. 14) due to melting. Possible melting of the crystalline component was also observed at 173° C. by hot stage microscopy.

The DVS results suggested that the material is less hygroscopic than that with low glycine loading. It exhibited a negligible weight change upon equilibration at 5% RH. However, a significant weight gain of approximately 74% was observed during the sorption step with the greatest amount gained between 75 and 95% RH. Similarly, a significant weight loss of 69% was observed in the desorption step (FIG. 15). Significant hysteresis was observed.

Water content in the sample was determined to be 1.87% by Karl Fischer titration.

The DSC thermogram of solid generated from 5.1 mg/mL EC145 in 3% glycine exhibited a glass transition event at 25.5° C. followed by a broad endotherm at 182.5° C. (FIG. 16).

In summary, solid dispersion of EC145 in glycine is determined to be partially crystalline at low glycine loading and crystalline at high glycine loading. The material with low glycine loading is more hygroscopic and contained more water than that with high glycine loading. Correspondingly, a lower T_{g} (24-26° C.) was observed in EC145 dispersions with low glycine loading (3%) and a higher T_{g} (40-43° C.) for dispersion with high glycine loading (10%) and the placebo sample. This might be a result of different water content. Because of the crystallization of glycine in the dispersion, the glass transition temperature determined from the DSC thermogram is only from a small fraction of the amorphous material in the dispersion. The T_{g} value is not a valuable criterion for miscibility evaluation of the dispersion. The IR spectrum attributed to EC145 can not be identified clearly in the dispersion because of low EC145 content and overlapping of IR signals from EC145 and the excipient.

Solid Dispersions with Mannitol

Four compositions were prepared with mannitol: a placebo sample with 3% mannitol, EC145 at 1.4 mg/mL in 3% and 10% mannitol, and EC145 at 5.2 mg/mL in 3% mannitol.

The DSC thermogram of the placebo sample exhibited a single glass transition event at 10.9° C. that is presumably attributed to amorphous mannitol. Two sharp endothermic events at above 140° C. are possibly due to the melt of the crystalline components.

The XRPD pattern of solid generated from 1.4 mg/mL EC145 in 3% mannitol exhibited some features that is indicative of crystalline phase and diffuse scattering. Diffuse scattering may be attributed to a separate amorphous phase, disordered/defective crystalline or nano-crystalline component.

The DSC thermogram of solid generated from 1.4 mg/mL EC145 in 3% mannitol exhibited a glass transition event at 30.4° C. followed by a major endothermic event at 143.6° C. Flow was observed at ˜140° C. by hot stage microscopy, possibly due to melting of the crystalline component.

The DVS results suggested that the material is very hygroscopic. The material exhibited a negligible weight change upon equilibration at 5% RH. However, a significant weight gain of approximately 81% was observed during the sorption step with the greatest amount gained between 75 and 95% RH. Similarly, a significant weight loss of 74% was observed in the desorption step with significant hysteresis (FIG. 17).

The water content in the sample was determined to be 4.52% by Karl Fischer titration.

The material containing 1.4 mg/mL EC145/10% mannitol is determined to be crystalline. The DSC thermogram exhibited two major endothermic events at 137.0 and 156.7° C. (FIG. 18) due to melting of the crystalline components. No glass transition event was observed. Evidence of flow was also observed at ˜132° C. and particle rounding was observed at ˜138° C. by hot stage microscopy.

The DVS results suggested that the material is less hygroscopic that with low mannitol loading. It exhibited a negligible weight change upon equilibration at 5% RH. However, a significant weight gain of approximately 57% was observed during the sorption step with the greatest amount gained between 75 and 95% RH. Similarly, a significant weight loss of 55% was observed in the desorption step (FIG. 19). Minor hysteresis was observed.

The water content in the sample was determined to be 1.92% by Karl Fischer titration.

The DSC thermogram of solid generated from 5.2 mg/mL EC145 in 3% mannitol exhibited a glass transition event at 15.5° C. followed by a broad endotherm at 142.7° C. (FIG. 20).

In summary, solid dispersion of EC145 in mannitol is determined to be partial crystalline at low manitol loading and to be crystalline at high mannitol loading. The material with low mannitol loading is more hygroscopic and contained more water than that with high mannitol loading. A T_{g} at ˜11° C. was observed in the placebo sample with mannitol, and a slightly higher T_{g} at ˜16° C. was observed in sample containing 5.2 mg/mL EC145 and 3% mannitol. The highest T_{g} (˜30° C.) was observed in sample containing 1.4 mg/mL EC145 and 3% mannitol. This might be a result of different EC145 concentration or different water content. Because of the crystallization of mannitol in the dispersion, the glass transition temperature determined from the DSC thermogram is only from a small fraction of the amorphous material in the dispersion. The T_{g} value is not a valuable criterion for miscibility evaluation of the dispersion. The IR spectrum attributed to EC145 can not be identified clearly in the dispersion because of low EC145 content and overlapping of IR signals from EC145 and the excipient.

Solid Dispersions with PEG400

Four compositions were prepared with PEG400: a placebo sample with 3% PEG400, EC145 at 1.4 mg/mL in 3% and 10% PEG400, EC145 at 5.3 mg/mL in 3% PEG400. Solid was obtained from dispersion containing 5.3 mg/mL EC145 and 3% PEG400. The other dispersion deliquesced during lyophilization.

Solid generated from 5.3 mg/mL EC145 was characterized by FTIR and mDSC. The DSC thermogram exhibited a major endotherm at 3.1° C. (FIG. 21), possibly due to the melt of ice. The IR spectrum displays features that are unique to this sample.

In summary, solid dispersion with PEG400 was not successful due to high moisture content.

Solid Dispersions with PVP10

Three compositions were prepared with PVP10: a placebo sample with 3% PVP10, EC145 at 1.4 mg/mL in 3% PVP10, and EC145 at 5.2 mg/mL in 3% PVP10. Dispersion with 20% PVP10 was not successful. EC 145 precipitated in 20% PVP solution.

The DSC thermogram of the placebo sample exhibited a glass transition event at 54.7° C. The T_{g} at 54.7° C. overlapped with a major endothermic event caused by evaporation. Therefore, the estimated T_{g} value may not represent the true value.

The sample containing 1.4 mg/mL EC145/3% PVP10 was determined to be X-ray amorphous. The XRPD pattern features three broad diffuse scattering maxima between 1 and 25 degree 2θ.

The DSC thermogram exhibited a glass transition event at 65.0° C. (FIG. 22). The T_{g} at 65.0° C. overlapped with a major endothermic event caused by evaporation. Therefore, the estimated T_{g} value may not represent the true value. The origin of those thermal events above 100° C. was unknown.

Flow was observed at ˜177° C. by hot stage microscopy, possibly due to decomposition.

The DVS results suggested that the material is very hygroscopic. The material exhibited a negligible weight change upon equilibration at 5% RH. However, a significant weight gain of approximately 121% was observed during the sorption step with the greatest amount gained between 75 and 95% RH. Similarly, a significant weight loss of 111% was observed in the desorption step with significant hysteresis (FIG. 23). The sample deliquesced upon completion of the test.

Water content in the sample was determined to be 3.52% by Karl Fischer titration.

The DSC thermogram of 5.2 mg/mL EC145/3% PVP10 exhibited a glass transition event at 50.0° C. (FIG. 24). The T_{g} at 50.0° C. overlapped with a major endothermic event caused by evaporation. Therefore, the estimated T_{g} value may not represent the true value. The origin of thermal events above 100° C. was unknown.

In summary, solid dispersion of EC145 in PVP10 is determined to be X-ray amorphous. The material is very hygroscopic. A weight change of 121% and 111% was observed in the sorption and desorption step, respectively. A T_{g} observed at 50-65° C. is possibly attributed to PVP10. Variation between the T_{g} values in different compositions is presumably a result of different water content in the samples. Due to low EC145 content in the samples and overlapping of IR signals from EC145 and the excipient, the IR spectrum attributed to EC145 can not be identified clearly.

Solid Dispersions with Sucrose

Four compositions were prepared with sucrose: a placebo sample with 3% sucrose, EC145 at 1.4 mg/mL in 3% and 20% sucrose, and EC145 at 5.2 mg/mL in 3% sucrose.

The DSC thermogram of the placebo sample exhibited a single glass transition event at 37.5° C. that is presumably attributed to amorphous sucrose.

Solid generated as 1.4 mg/mL EC145 in 3% sucrose was determined to be X-ray amorphous. The XRPD pattern features two broad diffuse scattering maxima at approximately 4 and 19 degree 2θ along with a shoulder at 15 degree 2θ.

The DSC thermogram exhibited two glass transition events at 53.1° C. and 123.4° C. (FIG. 25). The T_{g} at 53.1° C. overlapped with an endothermic event caused by evaporation. Therefore, the estimated T_{g} value may not represent the true value. Flow was observed at about 143° C. by hot stage microscopy.

The DVS results suggested that the material is very hygroscopic. The material exhibited a negligible weight change upon equilibration at 5% RH. However, a significant weight gain of approximately 127% was observed during the sorption step with the greatest amount gained between 75 and 95% RH. Similarly, a significant weight loss of 114% was observed in the desorption step with some hysteresis (FIG. 26). Sample deliquesced upon completion of the test.

Water content in the sample was determined to be 5.57% by Karl Fischer titration.

XRPD pattern of the solid generated as 1.4 mg/mL EC145 in 20% sucrose showed characteristics of an X-ray amorphous material and appeared similar to that in 3% sucrose.

The DSC thermogram exhibited a single glass transition event at 32.0° C. (FIG. 27), presumably attributed to amorphous sucrose. Flow was observed in areas at ˜100° C. by hot stage microscopy.

The DVS results suggested that the material is less hygroscopic that with low sucrose loading. It exhibited a negligible weight change upon equilibration at 5% RH. However, a significant weight gain of approximately 63% was observed during the sorption step with the greatest amount gained between 75 and 95% RH. Similarly, a significant weight loss of 53% was observed in the desorption step (FIG. 28). There was significant hysteresis during the desorption step. Sample deliquesced upon completion of the test.

The water content in the sample was determined to be 4.28% by Karl Fischer titration.

The DSC thermogram of the sample generated from 5.2 mg/mL EC145/3% sucrose is similar to that of a sample containing 1.4 mg/mL EC145 and 3% sucrose by featuring two glass transition events at 50.8° C. and 124.3° C. (FIG. 29). The T_{g} at 50.8° C. overlapped with an endothermic event caused by evaporation. Therefore, the estimated T_{g} value may not represent the true value.

In summary, solid dispersions of EC145 in sucrose are determined to be X-ray amorphous. The material with low sucrose loading (3%) is more hygroscopic and contained more water than that with high sucrose loading (20%). Two glass transition events, namely T_{g1} (51-53° C.) and T_{g2} (123-124° C.), were observed in samples containing EC145 and 3% sucrose. A single T_{g} at a lower temperature (32-38° C.) was observed in the placebo sample and an EC145 dispersion containing 20% sucrose. This might be a result of different sucrose concentration in the dispersion or potential interaction between EC145 and sucrose. Due to low EC145 content in the samples and overlapping of IR signals from EC145 and the excipient, the IR spectrum attributed to EC145 can not be identified clearly.

### Experimental Methods

### Lyophilization

For the dispersion screen, a concentrated EC145 at 214 mg/mL was prepared in water and diluted to a final concentration of 1.4 mg/mL in 50 mM citrate buffer, pH 6.2 in the presence of various excipients including glucose, glycine, mannitol, PEG400, PVP10, and sucrose. Two loadings of each excipient at 3 and 20% for glucose, PEG400, PVP10 and sucrose, or 3% and 10% for glycine and mannitol with a total of 12 compositions were prepared. Sample of EC145 at 5 mg/mL at one excipient concentration (3%) were also prepared. 6 placebo samples of excipient only at one concentration (3%) were generated for comparison to the solid dispersion compositions (containing EC145).

Total of 29 compositions were generated at approximately 200 mg to 1.2 g-scale, depending on the loading of the excipients. Approximately 6 mL of each composition was loaded into a 10-mL lyophilization vial. For excipients at 3% loading, each composition was prepared in duplicate.

EC145 solutions with different excipient at different concentration were frozen in a minus 80° C. freezer and loaded into a pre-chilled LABCONCO Freeze-Drier. Primary drying was conducted with shelf temperature setting at −55° C. for 140 hours, at −50° C. for 24 hours, at −45° C. for 24 hours, and at −20° C. for 25.5 hours. The secondary drying was conducted at 20° C. for 20.5 hours. Vacuum reading during lyophilization was about 0.06 Torr.

Sample vials were capped under vacuum inside the lyophilization chamber. The vials were sealed with parafilm immediately after removed from the freeze-drier.

### Sample Handling for Analytical Testing

To avoid moisture pick-up during multiple sampling, for each test, appropriate amount of sample was sub-sampled into each clean vial in a nitrogen bag under dim light.

### Instrumental Techniques

### Modulated DSC

MDSC data were obtained on a TA Instruments Q2000 differential scanning calorimeter equipped with a refrigerated cooling system (RCS). Temperature calibration was performed using NIST-traceable indium metal. The sample was placed into an aluminum DSC pan, and the weight was accurately recorded. The pan was covered with a lid and the lid was crimped. A weighed, crimped aluminum pan was placed on the reference side of the cell. Data were obtained using a modulation amplitude of ±1° C. and a 60 second period with an underlying heating rate of 2° C./minute from −50 to 200° C. The reported glass transition temperatures are obtained from the inflection point of the step change in the reversing heat flow versus temperature curve.

### Dynamic Vapor Sorption.Desorption (DVS)

Moisture sorption/desorption data were collected on a VTI SGA-100 Vapor Sorption Analyzer. NaCl and PVP were used as calibration standards. Samples were not dried prior to analysis. Sorption and desorption data were collected over a range from 5 to 95% relative humidity (RH) at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### FTIR

IR spectra were acquired on Nexus 670® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. Wavelength verification was performed using NIST SRM 1921b (polystyrene). An attenuated total reflectance (ATR) accessory (Thunderdome™, Thermo Spectra-Tech), with a germanium (Ge) crystal was used for data acquisition. Each spectrum represents 256 co-added scans collected at a spectral resolution of 4 cm⁻¹. The data acquisition parameters for each spectrum are displayed above the image in the Data section of this report. A background data set was acquired with a clean Ge crystal. A Log 1/R (R=reflectance) spectrum was obtained by taking a ratio of these two data sets against each other. Sample was analyzed neat on the Ge crystal.

Spectra analyses were conducted using OMNIC software. The second derivative spectra were generated using Norris derivative with segment length setting at 5 and gap between segments setting at 5.

### Hot Stage Microscopy

Hot stage microscopy was performed using a Linkam FTIR 600 hot stagewith a TMS93 controller on a Leica DM LP microscope equipped with a SPOT Insight™ color digital camera for acquiring images. Images were captured using SPOT software (v. 4.5.9). Temperature calibrations were performed using USP melting point standards. Samples were placed on a cover glass, and a second cover glass was placed on top of the sample. As the stage was heated, each sample was visually observed using a 20×0.4 N.A objective with crossed polarizers and a first order red compensator.

### Karl Fischer Titration

Coulometric Karl Fischer (KF) analysis for water determination was performed using a Mettler Toledo DL39 KF titrator. A blank titration was carried out prior to analysis. The sample was prepared under a dry nitrogen atmosphere and was extracted in approximately 1 mL dry Hydranal—Coulomat AD in a pre-dried vial. The supernatant was added to the KF coulometer through a septum and mixed for 10 seconds. The sample was then titrated by means of a generator electrode, which produces iodine by electrochemical oxidation: 2 I⁻→I₂+2e⁻. Two replicates were obtained to ensure reproducibility.

### XRPD

XRPD patterns were collected with a PANalytical X'Pert PRO MPD diffractometer using an incident beam of Cu radiation produced using an Optix long, fine-focus source. An elliptically graded multilayer minor was used to focus Cu Kα X-rays through the specimen and onto the detector. Prior to the analysis, a silicon specimen (NIST SRM 640d) was analyzed to verify the observed position of the Si 111 peak is consistent with the NIST-certified position. A specimen of the sample was sandwiched between 3-μm-thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data acquisition parameters for each pattern are displayed above the image in the Data section of this report including the divergence slit (DS) before the mirror and the incident-beam antiscatter slit (SS).

1. A solid pharmaceutical composition comprising EC145 and a bulking agent.

2. The composition of claim 1 wherein:
(a) the bulking agent comprises dextrose, glucose, glycine, inositol, mannitol, sorbitol, sucrose, a polyethyleneglycol (PEG), or a polyvinylpyrrolidine (PVP), or a combination thereof in an individual or combined range of about 3% to about 20% and/or arginine or proline in an individual or combined range of about 0.1 M to about 0.5 M; or
(b) the bulking agent comprises dextrose, inositol, mannitol, sorbitol or sucrose, or a combination thereof, in an individual or combined range of about 3% to about 6% and/or arginine or proline in an individual or combined range of about 0.1 M to about 0.5 M; or
(c) the bulking agent comprises about 3% to about 10% glycine or mannitol; or
(d) the bulking agent comprises about 3% to about 4% mannitol and 0% to about 1% sucrose; or
(e) the bulking agent comprises about 3% mannitol.

3. The composition of claim 1 comprising a further excipient.

4. The composition of claim 3 wherein the excipient comprises a buffer.

5. The composition of claim 4 wherein the buffer is an antioxidant which does not reduce a disulfide bond.

6. The composition of claim 4 wherein:
(a) the pH of the buffer is about 5.0 to about 8.0; or
(b) the pH of the buffer is about 5.7 to about 6.6; or
(c) the pH of the buffer is about 6.0 to about 6.6; or
(d) the pH of the buffer is about 6.2±0.2.

7. The composition of claim 4 wherein:
(a) the buffer comprises an ascorbate, sorbate, formate, lactate, fumarate, tartrate, glutamate, acetate, citrate, gluconate, histidine, malate, phosphate or succinate buffer; or
(b) the buffer comprises an ascorbate, lactate, tartrate, citrate, gluconate, malate, isocitrate or 2-hydroxybutyrate buffer; or
(c) the buffer comprises a citrate buffer.

8. The composition of claim 4 wherein:
(a) the concentration of the buffer is about 20 mM to 150 mM; or
(b) the concentration of the buffer is about 100 mM or is 100 mM; or
(c) the concentration of the buffer is about 50 mM or is 50 mM.

9. The composition of claim 4 wherein the buffer is a pH 6.2 citrate buffer.

10. The composition of claim 1 wherein:
(a) the solid corresponds to about 27 parts trisodium citrate dihydrate, about 1.5 parts citric acid, and about 40-80 parts mannitol to 2.8 parts EC145 by weight; or
(b) the solid corresponds to about 27 parts trisodium citrate dihydrate, about 1.5 parts citric acid, and about 60 parts mannitol to 2.8 parts EC145 by weight.

11. The composition of claim 1 wherein the solid is a lyophilized solid pharmaceutical composition.

12. The composition of claim 11 wherein the EC145 is X-ray amorphous.

13. The composition of claim 1 wherein the Raman spectrum of the solid comprises substantially the same spectrum as shown in FIG. 3 including a peak at about 1606 cm⁻¹.

14. The composition of claim 1 wherein the X-ray powder diffraction pattern of the solid comprises substantially the same pattern as shown in FIG. 2.

15. The composition of claim 4 which is a solid dispersion wherein the % weight increase at 65% relative humidity in dynamic vapor sorption.desorption does not exceed: (a) about 20% or 20%, or (b) about 10% or 10%, or (c) about 5% or 5%.

16. The composition of claim 1 which is a solid dispersion wherein, on a weight to weight dry basis, exclusive of residual water, the solid components correspond to:
(a) about 5-10 parts EC145, about 75-90 parts of a buffer and about 150-750 parts of a bulking agent; or
(b) about 8.6 parts EC145, about 81 parts trisodium citrate dihydrate, about 4.6-4.8 parts citric acid, and about 180 parts glycine; or
(c) about 8.6 parts EC145, about 81 parts trisodium citrate dihydrate, about 4.6-4.8 parts citric acid, and about 600 parts glycine; or
(d) about 8.6 parts EC145, about 81 parts trisodium citrate dihydrate, about 4.6-4.8 parts citric acid, and about 180 parts mannitol; or
(e) about 8.6 parts EC145, about 81 parts trisodium citrate dihydrate, about 4.6-4.8 parts citric acid, and about 600 parts mannitol.

17. The composition of claim 16 wherein the residual water content is about 1.5 to about 5% by weight.

18. A method of producing a lyophilized solid pharmaceutical composition comprising EC145 and a bulking agent as described in claim 1, and optionally further comprising a buffer, comprising lyophilizing an aqueous solution of EC145 and a bulking agent, wherein the solution optionally further comprises a buffer.

19. The method of claim 18, which method comprises one or more of the steps (i) and (ii):
completely freezing a liquid composition comprising EC145, a bulking agent as described in any of the embodiments herein, and optionally a buffer as described in any of the embodiments herein and an aqueous solvent at or below −20° C. prior to a primary drying step; and
(ii) the initial step of a primary drying stage comprising applying a vacuum to reduce the pressure effective to remove aqueous solvent from the frozen mixture of a liquid composition comprising EC145, a bulking agent as described in any of the embodiments herein, and optionally a buffer as described in any of the embodiments herein and an aqueous solvent, wherein the temperature is maintained at about −50° C. or below.

20. A lyophilized solid pharmaceutical composition comprising EC145 which is made by a process comprising lyophilizing a liquid composition comprising EC145, a bulking agent, an aqueous solvent and optionally a buffer.

21. The composition of claim 20 which is made by a process comprising one or more of the steps (i) and (ii):
(i) completely freezing the liquid composition comprising EC145, a bulking agent, an aqueous solvent and optionally a buffer at or below −20° C. prior to a primary drying step; and
(ii) the initial step of a primary drying stage comprising applying a vacuum to reduce the pressure effective to remove aqueous solvent from the frozen mixture of the liquid composition comprising EC145, a bulking agent, an aqueous solvent and optionally a buffer, wherein the temperature is maintained at about −50° C. for the first step of the primary drying.

22. The composition of claim 20 wherein:
(a) the bulking agent comprises dextrose, glucose, glycine, inositol, mannitol, sorbitol, sucrose, a polyethyleneglycol (PEG), or a polyvinylpyrrolidine (PVP), or a combination thereof in an individual or combined range of about 3% to about 20% and/or arginine or proline in an individual or combined range of about 0.1 M to about 0.5 M; or
(b) the bulking agent comprises dextrose, inositol, mannitol, sorbitol or sucrose, or a combination thereof, in an individual or combined range of about 3% to about 6% and/or arginine or proline in an individual or combined range of about 0.1 M to about 0.5 M; or
(c) the bulking agent comprises about 3% to about 10% glycine or mannitol; or
(d) the bulking agent comprises about 3% to about 4% mannitol and 0% to about 1% sucrose; or
(e) the bulking agent comprises about 3% mannitol.

23. The composition of claim 20 which comprises a buffer wherein:
(a) the pH of the buffer is about 5.0 to about 8.0; or
(b) the pH of the buffer is about 5.7 to about 6.6; or
(c) the pH of the buffer is about 6.0 to about 6.6; or
(d) the pH of the buffer is about 6.2±0.2.

24. The composition of claim 20 wherein the EC145 is X-ray amorphous.

25. The composition of claim 20 wherein the Raman spectrum of the solid comprises substantially the same spectrum as shown in FIG. 3 including a peak at about 1606 cm⁻¹.

26. The composition of claim 20 wherein the X-ray powder diffraction pattern of the solid comprises substantially the same pattern as shown in FIG. 2.

27. A drug product comprising a solid pharmaceutical composition comprising EC145 as described in claim 1 or 20.

28. The drug product of claim 27 further comprising an ampoule or a sealed vial.

29. The drug product of claim 28 further comprising a sealed vial.

30. The drug product of claim 27 wherein the pharmaceutical composition comprises a citrate buffer.

31. The drug product of claim 27 wherein the drug product is a multidose form.

32. The drug product of claim 27 wherein the drug product is a single dose form.

33. The dosage unit of claim 32 which provides on dilution or reconstitution with an aqueous diluent a solution comprising EC145 for intravenous administration as 2.0 mL of an aqueous sterile liquid formulation, which dosage unit contains 1.4 mg/mL of EC145.

34. The drug product of claim 27 wherein the EC145 is able to maintain a purity specification for EC145 of greater than or equal to 94% over the course of a year at ambient temperature (25° C.±2° C.).

35. A pharmaceutical composition obtained by reconstitution of a solid comprising EC145 and a bulking agent as described in claim 1 or 20.

36. The composition of claim 35 which composition comprises EC145 at a concentration of 1.4 mg/mL in an aqueous sterile liquid formulation the components of which comprise pH 6.2 citrate buffer, mannitol and water for injection.

37. A method of treating a patient with a tumor bearing functionally active folate receptors comprising at least one of the steps of:
(a) dissolving the solid pharmaceutical composition described in claim 1 or 20 in a pharmaceutically acceptable solvent to produce a pharmaceutically acceptable solution, and
(b) administering the solution to the patient in need thereof.

38. The method of claim 37, wherein the tumor is an ovarian tumor or a lung tumor.

39. The method of claim 38 wherein the tumor is an ovarian tumor.

40. The method of claim 39 wherein the tumor is a platinum-resistant ovarian tumor.

41. The method of claim 37 wherein the patient is further treated with pegylated liposomal doxorubicin or with doxorubicin which is not of the pegylated liposomal form.
